(19) United States
(12) Patent Application Publication
(10) Pub. No.: US 2003/0158671 A1
(43) Pub. Date: Aug. 21, 2003

(54) Systems and methods for predicting active site residues in a protein

(75) Inventor: Ketan S. Gajiwala, San Diego, CA (US)

Correspondence Address: PENNIE AND EDMONDS, 1155 AVENUE OF THE AMERICAS, NEW YORK, NY, 100362711

(73) Assignee: Structural Genomix, Inc.

(21) Appl. No.: 10/196,039

(22) Filed: Jul. 15, 2002

Related U.S. Application Data
(60) Provisional application No. 60/306,439, filed on Jul. 18, 2001.

(51) Int. Cl. G06G 7/48; G06G 7/58; G06F 19/00; G01N 33/48; G01N 33/50

(52) U.S. Cl. 702/19; 702/20; 703/11

(57) ABSTRACT

A system and method for predicting the active site residues in a target protein. The target protein, referred to interchangeably as the-target amino sequence or the target sequence, is pairwise aligned with a set of query sequences. Using these multiple pairwise sequence alignments, residue positions within the target sequence which satisfy specified sequence-based criteria are identified as candidate active site positions. Candidate active site positions are mapped to a three-dimensional representation of the target sequence and filtered against structure-based criteria. Positions within the target sequence that satisfy both the sequence-based and structure-based criteria are predicted to be active site residues.

### CROSS REFERENCE TO RELATED APPLICATION

Under 35 U.S.C. §119(e)(1), this application claims the benefit of prior U.S. provisional application No. 60/306,439, filed Jul. 18, 2001.

### 1. FIELD OF THE INVENTION

The present invention relates generally to bioinformatics, and particularly to a system and method for identifying the active site residues in a protein.

### 2. COMPUTER PROGRAM LISTING APPENDIX

One compact disc that includes a Computer Program Listing Appendix has been submitted in duplicate in the present application. The size of the files contained in the Computer Program Listing Appendix, their date of creation, their time of creation, and their names are found in Table 1 below.

TABLE 1
Contents of the Computer Program Listing Appendix

| | Date of | Time of | |
|---|---|---|---|
| Size | Creation | Creation | File Name |
| 1,804 | 06-25-011 | 7:23 p | allowed.pm |
| 24,164 | 06-25-01 | 7:23 pm | predact_patent.pl |

The Computer Program Listing Appendix disclosed in Table 1 is hereby incorporated by reference.

### 3. BACKGROUND OF THE INVENTION

The increase in the number of sequenced genomes is widening the gap between the number of known protein sequences and the number of proteins for which protein function is understood. The utility of the vast numbers of protein sequences derived from sequenced genomes depends largely on whether biological functions can be assigned to these protein sequences. Identifying specific residues forming the active site of the three-dimensional structure of the protein after it has folded into a physiologically relevant state greatly helps to understand the biological function of a protein. An active site is a site in a protein or peptide that associates with a substrate for protein activity, such as, for example, enzymatic activity. Active site residues form the active site of a protein. Identifying these active site residues is an important step in characterizing enzymatic reaction mechanisms that are facilitated by the protein. Additionally, identifying protein active site residues facilitates rational drug design, where inhibitors are designed to interact with the active site residues. It is expected that inhibitors that tightly interact with active site residues will inhibit some characteristics of the protein, including, but not limited to, enzymatic activity associated with the protein. Thus, predicting the protein active site has become a challenging problem in computational molecular biology (Irving et al., 2001, Proteins 42, 378-382).

To address the problem of identifying active site residues of a target protein, information about the biological function of proteins having sequences similar to the target protein sequence may be used. Further, if structural information is known for any of these similar sequences, the information can be used to make a homology model for the target protein. Sequence alignment is the most common method for determining which proteins are functionally similar to a target protein.

Additional methods exist in the art for identifying active site residues of a target sequence. In one approach, algorithms have been developed to use the structure of a target sequence in order to predict the enzyme class of the target sequence. For example, an algorithm called TESS has been developed to search for user-defined spatial combinations of atoms in the Protein Data Bank (PDB) (Wallace et al., 1997, Protein Science 6, 2308-2323). The PDB is a publicly available database of three-dimensional representations of proteins that have been derived by techniques such as two- and three-dimensional nuclear magnetic resonance as well as x-ray crystallography. TESS derives three-dimensional templates from three-dimensional representations deposited in the PDB. Using TESS, a new structure that corresponds to the target sequence is scanned against these three-dimensional templates in order to determine the active site residues of the target sequence.

In a similar method used to predict the active site residues from the three-dimensional representation corresponding to a target sequence, descriptors of protein active sites, termed “fuzzy functional forms” (FFFs), have been defined (Fetrow & Skolnick, 1998, Journal of Molecular Biology 281, 949-968). FFFs are derived from the geometry, residue identity, and conformation of protein active sites in known x-ray crystal structures of proteins. FFFs are used to identify the active sites residues in the ab initio or the threading models of various enzyme classes.

In yet another approach to determining the active site residues of a target sequence, a neural network based protocol is used to identify cavities on the surface of the target sequence. These cavities are considered potential active sites (Stahl & Schneider, 2000, Protein Engineering, 13, 83-99). The neural network approach has been applied to a set of 176 zinc metalloproteinases. In most, but not all cases, the actual active site residues of the target sequence were represented by one of the five largest cavities on the surface of the molecule.

In still another approach to identifying the active site residues of a target sequence, a computational tool called PASS has been developed. PASS characterizes regions of buried volume in target sequences following approaches similar to the neural network approach of Stahl & Schneider (Brady & Stouten, 2000, Journal of Computer-Aided Molecular Design 14, 383).

Still another approach to determining the active site residues of a target sequence is three-dimensional cluster analysis (Landgraf et al., 2001, Journal of Molecular Biology 307, 1487). Three-dimensional cluster analysis identifies active site residues by taking into account the conservation of spatially defined residue clusters within the target sequence relative to the target sequence as a whole.

In methods such as Goodford, 1985, J. Med. Chem. 28, 849-857, and Miranker, 1991, Proteins: Structure, Function, and Genetics 11, 29-34, interaction energies between the target protein and different probes are computed in an attempt to locate energetically favorable sites. Unfortunately, such energetic procedures require the assignment of proton locations and partial charges to the receptor atoms, which is not always a straightforward task. While van der Waals energies can indicate sterically available regions, the long-range nature of electrostatic potentials make the interpretation of energy levels difficult (e.g., a carboxylate in a binding site will emphasize positively charged probes even though negatively charged probes like the carbonyl oxygen may be part of the bound ligand.

Yet another approach to identifying active site residues of a target sequence is the use of geometric methods. Geometric methods require the three-dimensional coordinates of the target protein. Such methods explore the surface of the target protein without the use of energy models. There are a number of different types of geometric methods. Geometric methods include implementations found in the Molecular Operating Environment (MOE) Site Finder, which is distributed by the Chemical Computing Group (Montreal, Quebec Canada H3A 2R7), Ligsite (Hendlich et al., 1997, Journal of Molecular Graphics and Modeling 15, 359-363), the analytic geometric algorithms of Del Carpio et al., 1992, J. Mol. Graphics 11, 23-29, SURFNET (Laskowski, 1995, Journal of Molecular Graphics 13, 323-330), POCKET (Levitt, 1992, J. Mol. Graphics 10, 229-234), and the Cellular Logic Operations of Delaney (Delaney, 1992, J. Mol. Graphics 10, 174-177). Additional geometric methods include those disclosed by Kuntz et al., 1982, J. Mol. Biol. 161, 269-288, Ho and Marshall, 1990, J. Comput.-Aided Mol. Design 4, 337-354, and Bardford et al., 1988, Biochemistry 27, 6733-6741.

A direct method for determining active site residues of a target sequence is to solve the three-dimensional structure of the protein corresponding to the sequence complexed with a ligand that binds to the binding site of the protein. A number of proteins have been determined in such complexes using x-ray crystallographic or nuclear magnetic resonance techniques. The identity of the residues that form the binding site in a particular protein that has been solved by such techniques provides a source of information that can be used to predict which residues will form the binding site of another protein. To exploit this form of information, Stuart et al. (Stuart, 2001, LigBase: a database of families of aligned ligand binding sites in known protein sequences and structures, Bioinformatics 17, 1-2) developed a database that contains all binding sites of known structures. Furthermore, for each protein of known structure, the database provides an alignment with all related protein sequences and structures. The LigBase sequence alignments can be used to predict the binding site residues of proteins that are similar to proteins that have known complexed structures. However, the results identified by LigBase are not independently verified. Thus, for any given protein of interest, it is possible that the binding site identified by LigBase is not the real binding site. LigBase does not provide comprehensive methods for assessing the quality of the results obtained using LigBase. For example, if there is any error in the sequence alignment, LigBase will not identify the correct binding site.

In summary, known algorithms have identified active site residues in target sequences. However, theses known algorithms have drawbacks. Most known algorithms depend singularly on information derived from sequence alignments or on information derived from knowledge of the features of the three-dimensional representation of a target sequence. Yet singular reliance on sequence alignment information or on structural information fails to use all possible information available and potentially yields unsatisfactory results. For example, algorithms that depend exclusively on features of the three-dimensional representation, such as surface cavity shapes and/or residue solvent accessibility within the three-dimensional representation, fail to take into account useful information coded in sequence alignment information, such as residue conservation and substitution data. As used herein, the term “residue solvent accessibility” refers to the percent of the surface area of the residue that is exposed to solvent when the residue is part of a protein that is in a folded state. Algorithms that depend exclusively on sequence alignment data, on the other hand, fail to use important information that is available in a three-dimensional representation, such as residue solvent accessibility and residue proximity. Yet another disadvantage of active site residue identification algorithms is that they provide no filter to eliminate residues in the target sequence that do not participate in enzymatic reaction mechanisms facilitated by the target sequence.

Given the above background, what is needed in the art are improved algorithms for identifying the active site residues of target sequences.

### 4. SUMMARY OF THE INVENTION

The present invention predicts which residues in a target sequence are the active site residues. The system and method of the present invention uses both structural information and information derived from multiple pairwise sequence alignments to make these predictions. In the invention, a set of query sequences is aligned with the target sequence. A subset of aligned query sequences, in which each sequence in the set shares a high degree of similarity with the target sequence, is used in subsequent stages of the inventive method. In these subsequent stages, an alignment between the subset of highly similar query sequences and the target sequence is used to ascertain whether predetermined sequence-based criteria are satisfied at each residue position in the target sequence. Residue positions that satisfy the sequence-based criteria are considered candidate active site positions for the target sequence. Each candidate active site position is mapped to the three-dimensional representation of the target sequence in order to determine whether the position satisfies certain structure-based criteria. In this context, the term “mapping” means examining that portion of the three-dimensional representation of the target sequence that includes the candidate active site position. The three-dimensional representation of the target sequence represents the three-dimensional structure of the protein when the target sequence is in a physiologically relevant folded state. Candidate active site positions that satisfy the structure-based criteria are predicted to be the active site residues of the target sequence.

One aspect of the present invention provides a method for selecting a plurality of candidate active site positions in a target sequence. In the method, a plurality of query sequences is aligned one at a time with the target sequence to form a set of pairwise aligned query sequences. A subset of the aligned pairs of query sequences is chosen from this set of aligned query sequences. Each query sequence in the subset shares an overall sequence similarity with the target sequence that exceeds a default threshold sequence similarity. In one embodiment, the default threshold sequence similarity is an expectation value that indicates the likelihood that the sequence similarity between the target sequence and the aligned query sequence might occur by chance. For instance, in one implementation of the invention, the expectation value 1e−6 is used, where “1e−6” means that the probability that the observed alignment between the target and query sequence arises by chance is about one in a million for the database that is used for the alignment. The subset of aligned query sequences is used to determine whether sequence-based criteria are satisfied at each residue position in the target sequence. Residue positions satisfying the sequence-based criteria are considered candidate active site positions.

Each candidate active site position is identified by the residue type of the corresponding position in the target sequence. In one embodiment, the sequence-based criteria include: (i) requiring the residue at position i to be an allowed amino acid type, (ii) requiring each substitution type at position i in the subset of aligned sequences to be an allowed substitution type, and (iii) requiring that a threshold percentage of the subset of aligned sequences be aligned with position i of the target sequence. It will be appreciated that one embodiment of the present invention involves target sequences that are made from the twenty naturally occurring amino acids. As is widely appreciated in the art, amino acids may be referred to as residues when they are incorporated into a target sequence.

Each candidate active site position satisfying the sequence-based criteria is mapped onto a three-dimensional representation of the target sequence when it is in its physiological folded state, in order to determine whether the candidate active site position satisfies structure-based criteria. In one embodiment, the structure-based criteria consist of a single structural requirement. An exemplary structural requirement is that, when the residue type of a candidate active site position is mapped to the three-dimensional representation, a polar side-chain atom in the residue type must fall within a threshold distance of at least one other polar side-chain atom of a residue type of another candidate active site position mapped to the three-dimensional representation.

### 5. BRIEF DESCRIPTION OF THE FIGURES

Additional objects and features of the invention will be more readily apparent from the following detailed description and appended claims when taken in conjunction with the drawings, in which:

FIG. 1 is a diagram of a computer system with memory storing exemplary procedures and data of the present invention.

FIGS. 2A and 2B illustrate a process diagram of exemplary processing steps in accordance with one embodiment of the present invention.

FIG. 3 illustrates a process diagram of exemplary processing steps in accordance with an additional embodiment of the present invention.

FIG. 4 illustrates a process diagram of an algorithm used to automate an embodiment of the present invention.

Like reference numerals refer to corresponding parts throughout the several views of the drawings.

### 6. DETAILED DESCRIPTION OF THE INVENTION

### 6.1. Overview of the Invention

The present invention provides a system and method for predicting the active site residues in a target protein. Each query sequence in a set of query sequences is pairwise aligned with the target protein, also referred to as the target amino acid sequence or the target sequence. Using this multiple pairwise sequence alignment, residue positions within the target sequence satisfying specified sequence-based criteria are identified as candidate active site positions. Exemplary sequence-based criteria include requiring the residue type at the residue position to be an allowed residue type and requiring substitutions between corresponding positions in the target sequence and a query sequences to be allowed substitution types. Candidate active site positions are mapped to a three-dimensional representation of the target sequence and structure-based criteria are applied to the candidate active site positions. Positions within the target sequence that satisfy both the sequence-based criteria and the structure-based criteria are predicted to be active site residues.

One embodiment of the present invention is based on certain assumptions about the protein sequences and structures. These assumptions are that (i) residues not conserved in a functional family of proteins are not structurally or functionally important, (ii) functionally important residues are not tolerant of mutations while conserved positions important for the integrity of the structure are more tolerant to mutations, (iii) for enzymes, the set of functionally important residues must consist of polar/charged residues capable of participating in chemical reactions, and (iv) the set of functionally important polar/charged residues must cluster together in the three-dimensional representation of the protein. In one embodiment of the present invention, a residue at a particular position is considered not conserved in a functional family when it is not shared by a majority of the functional family members. Thus, consider the hypothetical residue “A” at position 10 in a first family member of a functional family of proteins. In this embodiment, the residue is not conserved if the majority of family members do not have an “A” at position 10.

### 6.2. Abbreviations and Definitions

Amino acid notations used for the twenty genetically encoded L-amino acids are conventional and are provided in Table 2.

TABLE 2
Contents of the Computer Program Listing Appendix

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless specified, one-letter and three-letter amino acid abbreviations designate amino acids in the L-configuration. Furthermore, polypeptide sequences presented as a series of one-letter and/or three-letter abbreviations are in the NH₂→COOH direction.

“Amino acid” refers to the twenty amino acids that are defined by genetic codons. The genetically encoded amino acids are glycine and the L-isomers of alanine, valine, leucine, isoleucine, serine, methionine, threonine, phenylalanine, tyrosine, tryptophan, cysteine, proline, histidine, aspartic acid, asparagine, glutamic acid, glutamine, arginine and lysine.

“Residue” refers to glycine and the L-isomers of the amino acids that are defined by genetic codons after they have been incorporated into the polypeptide chain of a protein.

“Polar amino acid” refers to a hydrophilic amino acid having a side-chain that is uncharged at physiological pH, but which comprises at least one covalent bond in which the pair of electrons shared in common by two atoms is held more closely by one of the atoms. Genetically encoded polar amino acids include Asn (N), Gln (Q), Ser (S), and Thr (T). Genetically non-encoded polar amino acids include the D-isomers of the above-listed genetically-encoded amino acids and homoserine (hSer).

### 6.3. Overview of System Components Used in the Invention

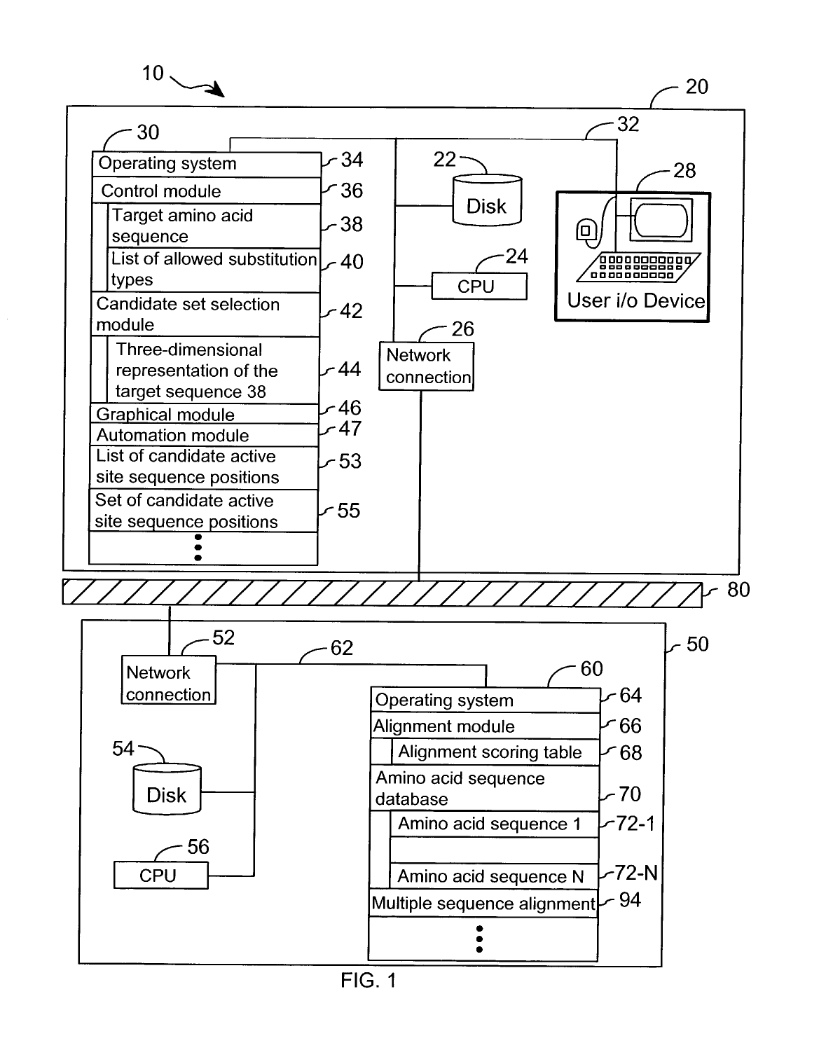

An exemplary system 10 in accordance with the present invention is illustrated in FIG. 1. In system 10, a target amino acid sequence 38 is identified on a first computer 20 and sent to a second computer 50 where the target amino acid sequence 38 is aligned against an amino acid sequence database 70. Sequences within the amino acid sequence database that share a high degree of sequence homology with the target sequence are returned to computer 20 as a multiple pairwise sequence alignment. Then, the multiple pairwise sequence alignment is used to apply sequence-based criteria to each position within the target amino acid sequence. Positions within the target amino acid sequence that satisfy these criteria are mapped onto a three-dimensional representation of the target amino acid sequence so that structure-based criteria may be applied.

System 10 includes computers 20 and 50 connected by transmission channel 80. Transmission channel 80 is any wired or wireless transmission channel. Computer 20 is any device that includes a central processing unit (CPU) 24 connected to a memory 30 and network connection 26 by a bus 32. Memory 30 preferably includes high-speed random-access memory (RAM) for the software modules and data structures of the instant invention.

In typical embodiments, computer 20 includes a main non-volatile storage unit 22, preferably a hard disk drive, for storing software and data. Typically, a portion of one or more of the software modules and/or data structures in memory 30 is stored in non-volatile storage unit 22. In preferred embodiments, computer 20 includes a user interface 28 which is capable of inputting and outputting a wide variety of data streams such as mouse commands, keyboard commands, graphics, and/or machine readable media back-up.

Operation of computer 20 is controlled primarily by control programs that are executed by CPU 24. The control programs are typically stored in memory 30. In a typical implementation, the programs and data stored in system memory 30 include:

an operating system 34;

a control module 36 for applying sequence-based criteria to a target sequence using a set of aligned sequences to identify a plurality of candidate active site positions in a target amino acid sequence;

a target amino acid sequence 38 for which active site residues are predicted;

a list of allowed substitution types 40 that may occur in a set of aligned sequences;

a candidate set selection module 42 for applying structure-based criteria to a plurality of candidate active site positions in order to identify a set of candidate active site positions from the plurality of candidate active site positions; and

a three-dimensional representation 44 of target sequence 38.

Furthermore, some embodiments of computer 20 include a graphical module 46 for viewing and evaluating the set of candidate active site residues 55 (FIG. 1) of target sequence 38. Commercial versions of module 46, include but are not limited to, Gaussian 92, revision C (Frisch, Gaussian, Inc., Pittsburgh, Pa. ©1992); AMBER, version 4.0 (Kollman, University of California at San Francisco, ©1994); QUANTA/CHARMM (Molecular Simulations, Inc., Burlington, Mass., ©1994); and Insight II/Discover (Biosym Technologies Inc., San Diego, Calif., ©1994).

In typical embodiments, computer 50 is a server that receives a target amino acid sequence 38 from computer 20 over transmission channel 80, aligns a database of amino acid sequences 70 against target amino acid sequence 38, and returns a multi-sequence alignment to computer 20 for subsequent processing steps in accordance with the present invention. In a typical implementation, therefore, computer 50 includes a bus 62 that interconnects CPU 56, memory 60, network connection 52, and non-volatile storage unit 54. Memory 60 preferably includes RAM for the software modules and data structures of the instant invention.

Operation of computer 50 is controlled primarily by control programs that are executed by CPU 56. The control programs are typically stored in memory 60. However, a portion of one or more of the software modules and/or data structures in memory 60 may be stored in non-volatile storage unit 54. In a typical implementation, the programs and data stored in system memory 60 include:

an operating system 64;

an alignment module 66 for aligning an amino acid sequence database to target amino acid sequence 38;

an alignment scoring table 68 for calculating the degree of similarity between target amino acid sequence 38 and an amino acid sequence in an amino acid sequence database; and

an amino acid sequence database 70, which includes one or more amino acid sequences 72.

### 6.4. Processing Steps in Accordance with One Embodiment of the Invention

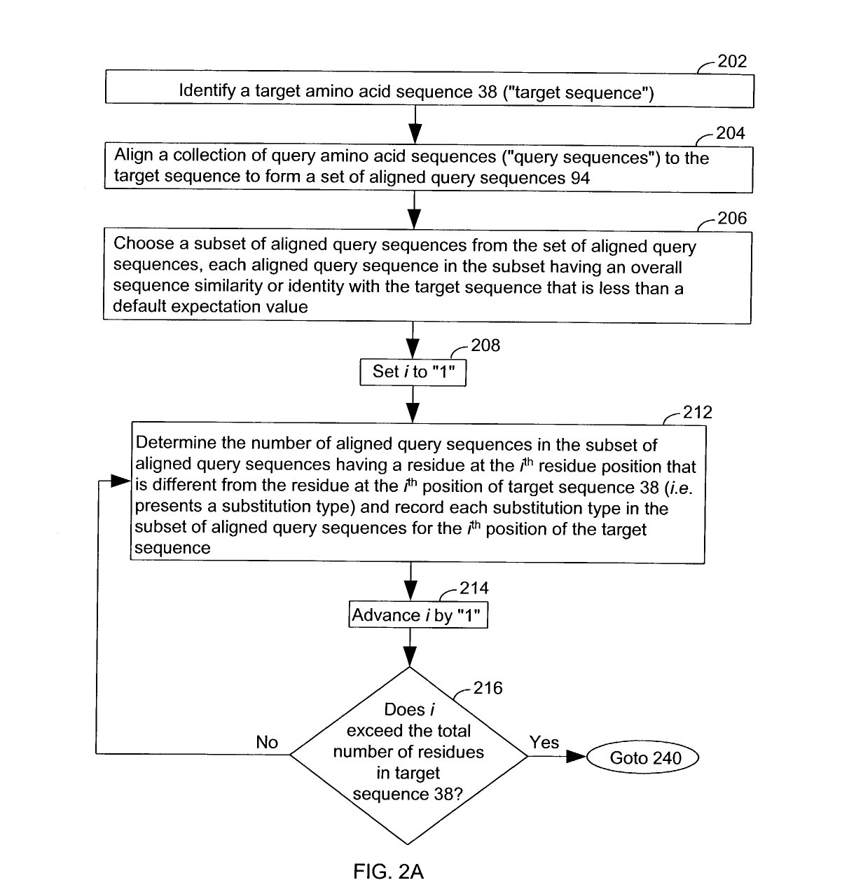

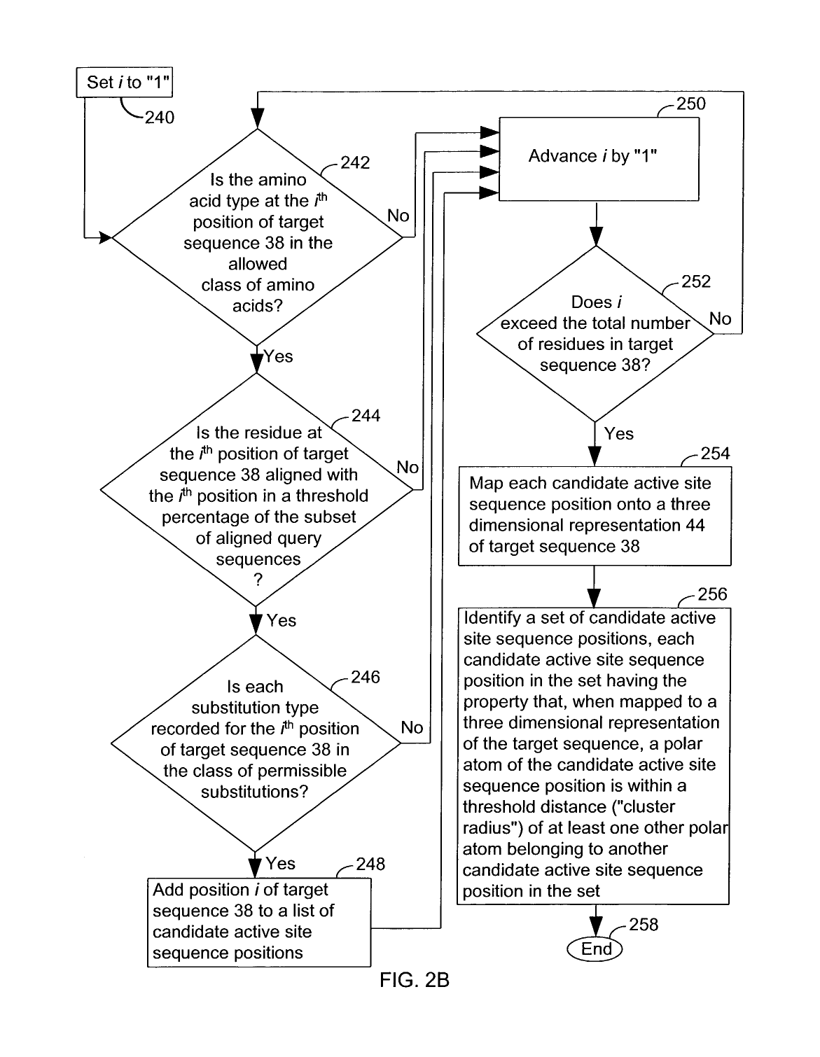

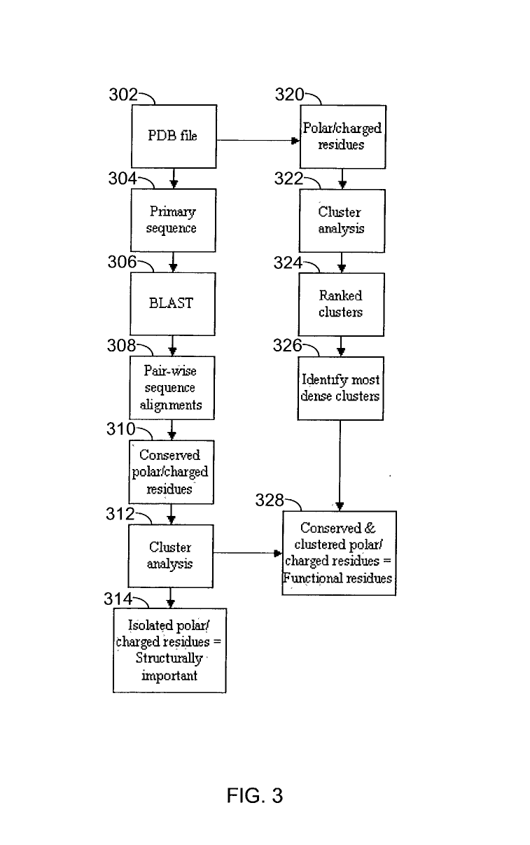

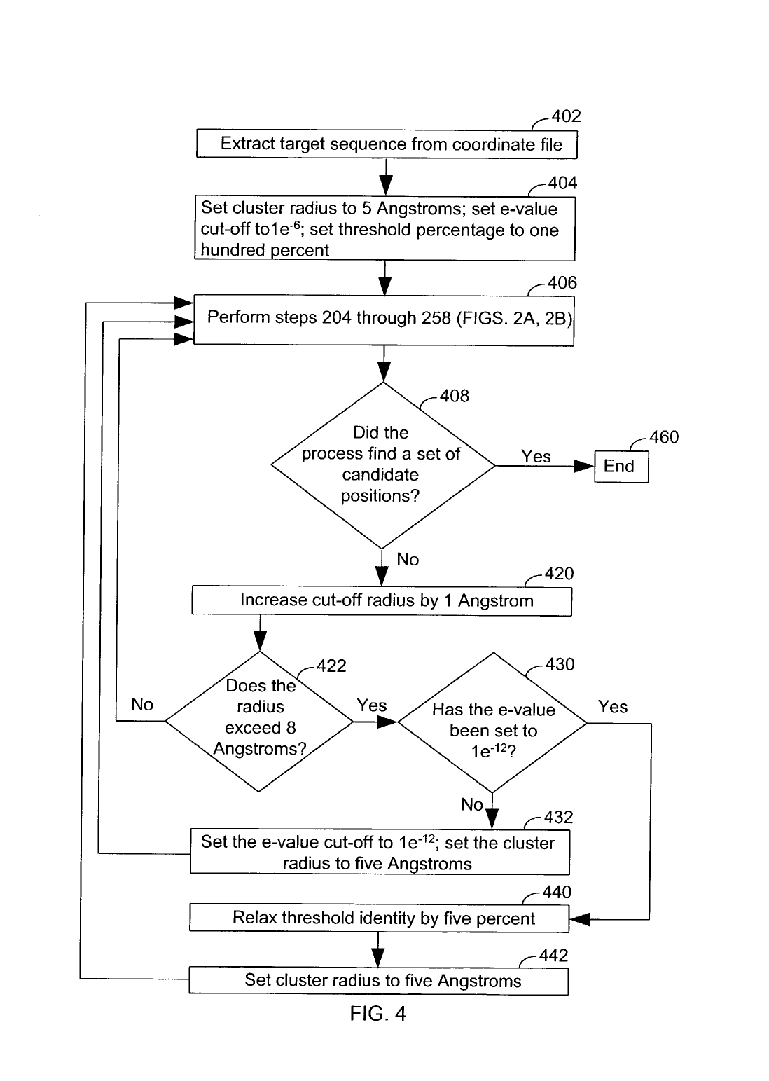

The function of the various software modules and data structures of system 10 will now be described in more detail using the flowchart presented in FIGS. 2A and 2B, which summarizes processing steps in accordance with one embodiment of the present invention.

Processing step 202. In processing step 202, a target amino acid sequence 38 is selected. In one embodiment of the present invention, a target amino acid sequence 38 is selected by extracting a sequence from the “SEQRES” records of a PDB file. A PDB file provides a method for recording information about a protein, including the sequence of the protein and atomic coordinates that represent the three-dimensional structure of the protein (Wallace et al., 1997, Protein Science 6, 2308-2323). The present invention imposes no requirements on the format of target amino acid sequence 38 provided that the sequence is machine-readable. In a typical embodiment, target amino acid sequence 38 is in FASTA format. A sequence in FASTA format begins with a single-line description followed by lines of amino acid sequence data. The description line is distinguished from the sequenced data by a greater-than (“>”) symbol in the first column. An example sequence in FASTA format is provided in Table 3.

TABLE 3
Exemplary target amino acid sequence 38 data
format

>gi|532319|pir|TVFV2E|TVFV2E envelope protein
ELRLRYCAPAGFALLKCNDADYDGFKTNCSNVSVVHCTNLMNTTVTTG.L
LNGSYSENRTQIWQKHRTSNDSALILLNKHYNLTVTCKRPGNKTVLPVTI
MAGLVFHSQKY-LRLRQAWC

In the exemplary data format of Table 3, sequences are represented in the standard amino acid codes of Table 2, lower-case letters that represent individual amino acids are mapped to upper-case, and a single hyphen or dash is used to represent a gap of indeterminate length in the amino acid sequence. For more details on the exemplary data format of Table 3, see (http://www.ncbi.nlm.nih.gov/BLAST/fasta.html).

Processing step 204. In processing step 204, each query amino acid sequence in a collection of amino acid sequences (e.g., a database of amino acid sequences) is aligned to the target amino acid sequence identified in processing step 202. As illustrated by system 10 (FIG. 1), the alignment of a collection of query amino acid sequences stored in an amino acid sequence database to the target amino acid sequence may occur on a remote computer. In fact, there are a number of publicly available resources for performing processing step 204. One such public resource may be found at http://www.ncbi.nlm.nih.gov/BLAST.

One of skill in the art will appreciate that the alignment performed in processing step 206 (FIG. 2A) may be performed by any of several different sequence alignment algorithms. In some embodiments of the present invention, the sequence alignment algorithm is coded by alignment module 66 (FIG. 1). Representative sequence alignment algorithms are disclosed in subsection 6.6, infra.

In some embodiments of the present invention, the alignment between the primary sequence of the target amino acid sequence and the primary sequence of a query amino acid sequence in a database of amino acid sequences 70 (FIG. 1) is determined by an iterated profile search method. One example of an iterated profile search method comprises comparing the primary sequence of the target amino acid sequence 38 to a protein database 70 using a basic local alignment search tool. See Altschul et al., 1990, J. Mol. Biol. 215, 403-410; and Karlin et al., 1993, PNAS USA 90, 5873-5787. This comparison results in a multiple sequence alignment 94 (FIG. 1) and a profile from significant local alignments found in the comparison of the primary sequence of the target amino acid sequence to query amino acid sequences in the protein sequence database. Then, the profile is compared to the query amino acid sequences in the protein database 70 using the basic local alignment search tool in order to improve the profile. The profile is revised to include significant local alignments found in the comparison of the profile to the protein database. The steps of comparing the profile to the amino acid sequences in the protein database using the basic local alignment search tool and using this comparison to improve the profile are repeated a fixed number of times or until the profile converges.

In some embodiments of the present invention, only those proteins in the amino acid sequence database 70 used in processing step 204 that share a predetermined amount of sequence identity and/or sequence similarity are used to form the multiple sequence alignment 94 and/or a profile. In some embodiments of the present invention, only those proteins in the database of proteins used in processing step 204 that have an expectation score that is within a predetermined range are used to form the multiple sequence alignment 94 (FIG. 1) and/or a profile. Section 6.7, infra, defines the terms sequence identity and sequence similarity and quantifies the amount of sequence identity and/or the amount of sequence similarity that a query protein sequence must have in relation to the target (first) protein sequence in order to be included in sequence alignment 94, in accordance with some embodiments of the present invention. Furthermore subsection 6.8, infra, describes expectation values and how they are used to determine which proteins in the database used in processing step 204 are included in the multiple sequence alignment, in accordance with some embodiments of the present invention.

Processing step 206. In processing step 206, the set of aligned query sequences is filtered so that only those pairings of sequences that achieved scores that satisfy a predetermined criterion are selected. The predetermined criterion used may be, for example, a degree of similarity, an expectation value (see Section 6.8, infra), a percent degree of similarity and/or a degree of identity (see Section 6.7, infra).

An expectation value is a measure of the likelihood that an alignment between two sequences might occur by chance in a given database of sequences. Thus, in embodiments in which the predetermined criterion is a default expectation value, query sequences 72 having an expectation value with the target amino acid sequence 38 that is less than about 1e−2 to about 1e−9 are selected for further processing. The expectation value range 1e−2 to 1e−9 includes any alignment between a target and query sequence in which the likelihood that such an alignment would occur by chance is in the range from about 1 in 100 to about 1 in 10⁹. In another embodiment, aligned sequences 72 having an expectation value that is less than about 1e−5 are selected from the database of query sequences 70. That is, alignments having a likelihood of occurring by chance that is 1 in 10⁵ or smaller are selected. In yet another embodiment, aligned sequences 72 from the database of query sequences 70 having an expectation value that is less than about 1e−7 (one in ten million) are selected. In a preferred embodiment, aligned sequences 72 from the database of query sequences 70 having an expectation value that is less than about 1e−6 (one in a million) are selected.

Processing steps 208-216. Processing steps 208-216 apply predetermined sequence-based criteria to the subset of aligned query sequences chosen in processing step 206. The sequence-based criteria are applied by comparing corresponding individual residue positions in the subset of aligned query sequences and the target amino acid sequence 38. Individual residue positions are referred to herein as positions. As will be explained in detail for individual processing steps, any number of sequence-based criteria are applied to each residue position in a target amino acid sequence. Exemplary criteria include: (i) requiring only predetermined substitution-types to occur between the specified position in the target sequence 38 and corresponding positions in the subset of aligned sequences; (ii) requiring that the amino acid type at the specified position in the target sequence 38 be an allowed type; and (iii) requiring that a threshold percentage of the subset of aligned query sequences include a residue that aligns with the specified position in target sequence 38.

Processing step 208. In processing step 208, the residue position index i is initialized to the value “1”.

Processing step 212. In processing step 212, the number of aligned query sequences in the subset of aligned query sequences having a residue at the i^{th} position that is different from the residue at the i^{th} position of target sequence 38 is recorded as substitution types. For each of these recordations, the substitution type is noted. Substitution types are described with reference to Table 4. In Table 4, the amino acid sequence of target sequence 38 is set forth in the first row of column 2. Subsequent rows list aligned amino acid sequences identified in processing step 206.

TABLE 4
Exemplary comparison of a target sequence 38 to a
subset of aligned query sequences

| | |
|---|---|
| Target sequence 38 | ELRLRYCA |
| Aligned sequence 1 | ALRLRYCA |
| Aligned sequence 2 | QLRL..CA |
| Aligned sequence 3 | NLRLRKCA |

As an example, in the case where i is set to “1” three substitution types for the dataset of Table 4 are recorded for position “1”. These substitution types are E→A (target amino acid sequence 38 to aligned sequence 1), E→Q (target amino acid sequence 38 to aligned sequence 2), and E→N (target amino acid sequence 38 to aligned sequence 3). In this notation, the amino acid before the “→” refers to the target amino acid and the amino acid after the “→” refers to the query amino acid. Furthermore, for the dataset of Table 4, where i is set to “2”, no substitution types are recorded because all three aligned sequences and the target sequence have an identity of “L” at this position.

Processing step 214. In processing step 214, the counter i is advanced so that the next sequential residue position in target amino acid sequence 38 may be examined for substitution types.

Processing step 216. Processing step 216 tests if i exceeds the total number of residues in target sequence 38. When i exceeds the total number of residues in target sequence 38 (216-Yes), control passes to processing step 240 where additional sequence-based criteria are applied to residue positions in target sequence 38. If i does not exceed the total number of residues in target sequence 38 (216-No) control passes back to processing step 212 where position i in target sequence 38 is examined. As an example, in Table 4, exemplary target sequence 38 has eight residues. Thus, when i advances to 9, control passes to processing step 240.

Processing step 240. In processing step 240 (FIG. 2B), the counter i is reset to “1” so that application of additional sequence-based criteria may be applied to each residue in target sequence 38.

Processing step 242. In processing step 242, a determination is made as to whether the identity of the residue at position i of target sequence 38 is in an allowed class of amino acid types. In a preferred embodiment, the allowed class of amino acid types consists of R, K, H, D, E, S, T, and C because these residues can participate in enzymatic reactions that take place in the active site of a protein. So, in the exemplary target sequence of Table 4, positions 1 (“E”), 3 (“R”), 5 (“R”), and 7 (“C”) are in the allowed class of amino acids (242-Yes) whereas all remaining positions in the exemplary target sequence 38 are not in the allowed class (242-No).

Processing step 244. In processing step 244, a determination is made as to whether the amino acid type at the i^{th} position of the target sequence is aligned with a threshold percentage of residues at the i^{th} position in the subset of aligned query sequences. In one embodiment, when a threshold percentage of the set of aligned query sequences have an amino acid in a corresponding position (244-Yes), control passes to processing step 246. If not (244-No), control passes to processing step 250. In one embodiment, when a threshold percentage of the aligned sequences have a residue, of any type, that corresponds to position i in target sequence 38, this sequence-based criterion is satisfied. Thus, for example, in Table 4, all three aligned sequences have a residue that corresponds to position “1” of the target sequence 38. Thus, processing step 244 will return a value of 100 percent for position “1”. In contrast, at positions “5” and “6” of target sequence 38, only two out of three sequences have corresponding residues because aligned sequence two has a gap at these positions. Thus, processing step 244 will return a value of 66 percent for positions “5” and “6”. In some embodiments, this sequence-based criterion is only satisfied when a threshold percentage of the aligned sequences have the same exact residue type at position i as that at position i of the target sequence 38. In embodiments in which the sequence-based criterion requires an absolute match of, for example, 100 percent, positions 2, 3, 4, 7 and 8 in Table 4 would satisfy the criterion.

In one embodiment of the present invention, the threshold percentage of the subset of aligned query sequences that must have a residue at corresponding position i is thirty percent or greater. In another embodiment, the threshold percentage of the subset of aligned query sequences that must have a residue at corresponding position i is fifty percent or greater. In yet another embodiment, the threshold percentage of the subset of aligned query sequences that must have a residue at corresponding position i is seventy percent or greater. In still another embodiment, the threshold percentage of the subset of aligned query sequences that must have a residue at corresponding position i is eighty percent or greater. In one instance, the threshold percentage is about eighty percent.

Processing step 246. In processing step 246, a determination is made as to whether each substitution type recorded for position i in target sequence 38 is an allowed substitution type. In a first embodiment of the present invention, the allowed substitution types consist of R→K, K→R, K→H, H→K, H→R, R→H, D→E, E→D, E→H, H→E, D→H, H→D, S→T, and T→S. In this notation, the first amino acid designation refers to the residue type in a position in the target sequence 38 and the second amino acid designation refers to the residue type in a corresponding position in an aligned sequence. Various other embodiments of the present invention have a smaller number of substitution types and, in particular, the set of substitution types may be any possible subset of the set of substitution types of the first embodiment of the present invention. In still other embodiments, additional substitution types, such as C→S or S→C, are allowed.

Some embodiments of the present invention impose the additional sequence-based criterion that there be a maximum of about five different substitution types at any given sequence position i. Thus, in such embodiments, target sequence positions 38 that include more than five allowed substitution types will not be considered active site residues of the target protein. Other embodiments of the present invention require that there be a maximum of two different allowed substitution types at any given position i in the target sequence. If each substitution type recorded for position i is allowed and the total number of different substitution types is less than a predetermined number (246-Yes), control passes to processing step 248. If not (246-No), control passes to processing step 250.

Processing step 248. For any given residue i in a target sequence 38, when control is passed to processing step 248, the position i has satisfied the sequence-based criteria of the instant invention and the position i is added to a list of candidate active site positions 53 (FIG. 1).

Processing step 250. Processing step 250 advances i by “1” so that sequence-based criteria are applied to each residue in target sequence 38 and so that the list of candidate active site sequence positions 53 includes all possible candidates.

Processing step 252. Processing step 252 returns control to processing step 242 (252-No) if i has not reached the end of target sequence 38. Control is passed to processing steps 254-256 (252-Yes), where structure-based criteria are applied, if the end of target sequence 38 has been reached in the 242-252 processing loop.

Processing step 254. In step 254, each candidate active site sequence position is individually mapped to a three-dimensional representation 44 of target sequence 38. For example, if residue position 5 of target sequence 38 is in the list of candidate active site sequence positions 53 (FIG. 1) identified in successive instances of step 248 (FIG. 2B), the coordinates of residue position five in a three-dimensional representation 44 of target sequence 38 are considered in processing step 256. In one embodiment of the present invention, processing step 254 comprises (i) loading a molecular representation 44 of target sequence 38 and, (ii) building a data structure that includes an identification of each residue in the molecular representation that is in the list of candidate active site sequence positions 53 (FIG. 1) identified by instances of processing step 248 (FIG. 2B).

The three-dimensional representation 44 of target sequence 38 may be generated or derived from any number of sources. Such sources include, for example, atomic resolution crystal structures of the target sequence, a model of the target sequence derived by nuclear magnetic resonance, and/or a homology model of target sequence 38 created using modeling software.

Processing step 256. Processing step 256 sequentially considers each polar/charged atom in the list of candidate active site sequence positions 53 (FIG. 1) that was mapped to a three-dimensional representation 44 of target sequence 38 by processing step 254. In one embodiment, this consideration is implemented in accordance with the pseudocode of Table 5.

TABLE 5
Exemplary pseudocode for mapping a candidate active site position
to a three-dimensional representation 44 of target sequence 38

```
500 For each residue i in a list of candidate active site positions {
502  For each polar/charged side-chain atom N in residue i {
504  Search for any residue j in the list of candidate active site
        positions that has a polar or charged side-chain atom M that
        is within a threshold distance Q of polar or charged atom N;
506  If M exists, add residue i to a set of candidate active
        site positions;
508  } /* for polar/charged atom N */
510 } /* for residue I */
```

Line 500 of the exemplary pseudocode of Table 5 ensures that structure-based criteria are applied to each residue i in the list of candidate active site sequence positions 53 (FIG. 1) that was built by successive instances of processing step 248 (FIG. 2B).

Line 502 examines each polar/charged side-chain atom N of each residue i in the list of candidate active site positions 53. In one embodiment of the present invention, polar/charged atoms N are any atoms within a residue that may be designated as “OD1”, “OD2”, “OE1”, “OE2”, “NZ”, “NE”, “NH1”, “NH2”, “ND1”, “NE2”, “SG”, “OH”, “OG”, “OG1”, “ND2”, and “NE2” when the standard Brookhaven nomenclature described in Table 6 is used.

TABLE 6
Standard Brookhaven format for atom type designation

| | |
|---|---|
| First letter: | Atomic symbol of the atom, “C” for carbon, |
| | “S” for sulfer, “O” for oxygen |
| Second letter | Side-chain distance abbreviation for the atom, |
| | which is, in order of increasing distance from the |
| | main-chain, “B” for beta, “G” for gamma, |
| | “D” for delta, “E” for epsilon, |
| | “Z” for zeta, and “H” for eta |
| Tailing number, | Identifies the branch direction of the branch |
| if any | that the atom is on |

Line 504 searches for any residue j in the list of candidate active site positions 53 that has a polar atom M that is within a threshold distance of a polar atom N. To illustrate this concept, consider the case of two residues 100 and 110 of an exemplary target protein 38 that have satisfied the sequence-based criteria of the present invention and are in the list of candidate active site sequence positions 53 (FIG. 1) identified by processing step 248 (FIG. 2B). If residue 100 includes an atom N of the type OE1 (“100:OE1”), the atom will be selected by an instance of line 502 and any atom M of any residue j in the list of candidate active site positions 53 that falls within a threshold distance Q of 100:OE1 will be identified by line 504 of the pseudocode. For example, if residue 110 has an atom N of the type NZ (“110:NZ”) and 110:NZ is within a threshold distance of 100:OE1, M exists and residue 110 will be added to the set of candidate active site positions 55 (FIG. 1) that are predicted to compose the active site of the protein. In this example, the nomenclature xxx:atom_type is used, where xxx refers to the residue position and atom_type refers to the atom type in accordance with Table 6.

In some embodiments of the present invention, the threshold distance Q that is used in line 504 is seven angstroms or less. In other embodiments of the present invention, the threshold distance Q that is applied is six angstroms or less. In even more preferred embodiments the threshold distance Q that is applied in line 504 is five angstroms or less. In yet another embodiment, the threshold distance Q is selected from the range of about 2.0 A to about 7.0 Å.

Processing step 258. In processing step 258 the algorithm ends with the prediction that the set of candidate active site sequence positions 55 (FIG. 1) form the active site of target amino acid sequence 38.

### 6.5. An Alternative Embodiment of the Invention

In FIG. 1, exemplary system 10 includes computers 20 and 50. In such an embodiment, computer 50 is typically a server that is used to align a target amino acid sequence 38 against an amino acid sequence database 70. However, it will be appreciated that the software modules and data structures of the present invention may be on the same computer or distributed across any number of computers, so long as the software modules and data structures are machine accessible using transmission channel 80.

In FIG. 3, another embodiment of the present invention is illustrated. This embodiment consists of two parts, a sequence analysis and a three-dimensional structure analysis.

Sequence analysis looks at the invariant/highly conserved polar residues of all the sequences that are similar to that of the target sequence 38. Once these residues are identified, they are examined in the context of their positions on the three-dimensional representation 44 of the target sequence 38. The conserved positions that cluster together are hypothesized as the functionally important sites while the isolated conserved positions are annotated as of structural significance.

The structure analysis looks at every single side-chain polar and/or charged atom and the number of contacts it makes with the other polar and/or charged atoms in its neighborhood. Thus, each atom is at the center of a cluster of atoms with which it makes contact. The largest of these clusters are suggested as the potential active sites.

In processing step 302 (FIG. 3), the three-dimensional representation 44 for target sequence 38 is read. In one embodiment of the present invention, the three-dimensional representation is in Brookhaven PDB format. In processing step 304, the primary sequence of target sequence 38 is extracted from the three-dimensional representation. When the three-dimensional representation is in Brookhaven PDB format, for example, the primary sequence of target sequence 38 is read from the SEQRES records of the PDB file. In processing step 306, the sequence information is converted into one-letter codes in accordance with Table 2 and used as the query sequence 38 for a sequence alignment program such as BLAST (Altschul et al., 1997, Nucleic Acids Research 25, 3389-3402, 1997). In one embodiment, BLOSUM62 is the alignment scoring table 68 (FIG. 1) used for the scoring of sequence similarities in processing step 306. In yet another embodiment, the default expectation value (E-value) cutoff for the search in processing step 306 is about 1e−6, but this parameter is readily changed depending on the experimental circumstances.

In processing step 308, the output of processing step 306 is analyzed as a set of pairwise sequence alignments. For each of the alignments, processing step 310 records the number of conservations, substitutions and the type of substitutions at each residue position of target sequence 38. These numbers, when tallied to give the number of times the residue is conserved/substituted (and the types of substitutions), reflect the variability of a residue at the particular positions in target sequence 38. In one embodiment, the sequence analysis is tabulated in processing step 310 with columns for the individual residue position, number of conservations, number of substitutions and the types of substitutions. The residue positions are sorted first based on the most conserved residues and then based on the least number of different types of substitutions. A sample line of output is provided in Table 7.

TABLE 7
Sample output generated in processing step 410
Of all the potentially active residues, in 45 pairwise
alignments with E value = 1e−6:

| Residue | conserved | substituted | . . . times . . . to |
|---|---|---|---|
| 59 S | 21 | 22 | 20T 1K 1- |

Table 7 translates to: the position Ser59 of target sequence 38 has been conserved 21 times, substituted 22 times (to T—20 times, K—once and deleted once), and not aligned two times. Here, the amino acid type and the position type are fused together to identify the position and residue type in target sequence 38 (e.g. Ser59). In one embodiment of the present invention, the most highly conserved residues occur at the top of the tabulated list of residue positions. In processing step 310, polar/charged residues are chosen as the potential active site residues.

In order to be chosen as a residue of potential significance in one embodiment of the present invention, each residue has to fulfill the following criteria (i) it should belong to the following list of residues: R, K, H, D, E, S, T and C; (ii) it should be aligned in at least about eighty percent of the total number of pairwise alignments generated in processing step 308; and (iii) when the position is substituted, the following substitutions are allowed: R⇄K⇄H; D⇄E⇄H; or S⇄T.

The positions fulfilling the criteria imposed in processing step 310 are of potential structural and/or functional significance. In processing step 312, these positions are then mapped to three-dimensional representation 44 of target sequence 38 and analyzed using cluster analysis. The subset of residues that are within a user-defined distance from each other is suggested as the most likely functionally important region of molecule in processing step 314.

In a parallel analysis of the three-dimensional representation 44 of the target sequence 38 (processing steps 320-326), the focus is on the polar atoms of the amino acid side-chains. In processing step 320, the number of contacts made by each of these atoms with the other side-chain polar atoms is determined using the contact distance provided by the user. In one embodiment of the present invention, this contact distance is about 4.7 Å to about 5.3 Å. In turn, the number of polar contacts made by each polar side-chain is calculated (processing step 322) and the residue positions are sorted based on the number of contacts (processing step 324). The largest of these clusters is then proposed to be of potential functional importance (processing step 326).

### 6.6. Representative Sequence Alignment Algorithms

Existing sequence comparison processes used in some embodiments of processing step 204 (FIG. 2A) may be divided into two main classes: global comparison methods and local comparison methods. In global comparison methods, a pair of sequences is aligned in its entirety and scored in a single operation (Needleman and Wunsch, 1970, Molecular Biology 48, 443). In local comparison methods, only highly similar segments of two sequences are aligned and scored; and a composite score is computed by combining the individual segment scores, e.g., the FASTA method (Pearson and Lipman, 1988, Proc. Natl. Acad. Sci. USA 85: 2444-2448), the BLAST method (Altschul, et al., 1990, Journal of Molecular Biology 215, 403-410; Altschul et al., 1997, Nucleic Acids Research 25, 3389-3402) and the BLAZE method (Brutlag, et al., 1993, Computational Chemistry 17, 203-207).

In some embodiments, sequence alignment between the target amino acid sequence and query amino acid sequence that is performed in processing step 204 (FIG. 2A) is determined using an algorithm such as Basic Local Alignment Search Tool (BLAST), PSI-BLAST, PHI-BLAST, WU-BLAST-2, and/or MEGABLAST. See Altschul et al., 1990, J. Mol. Biol. 215, 403-410; Altschul et al., 1996, Methods in Enzymology 266, 460-480; and Karlin et al., 1993, PNAS USA 90, 5873-5787.

Additional algorithms that may be used to align the target amino acid sequence 38 to the primary sequence of each query amino acid sequence in a database include FASTA (Pearson, 1995, Protein Science 4, 1145-1160), ClustalW (Higgin et al., 1996, Methods Enzymol. 266, 383-402), DbClustal (Thompson et al., 2000, Nucl. Acids Res. 28, 2910-2926), and the Molecular Operating Environment (Chemical Computing Group, Montreal, Quebec Canada H3A 2R7).

The various multiple protein sequence alignment formats supported by alignment module 60 include, but are not limited to, FASTA (Pearson, 1995, Protein Science 4, 1145-1160), ClustalW (Higgin et al., 1996, Methods Enzymol. 266, 383-402), MSF (European Molecular Biology Laboratory, Meyerhofstr. 1, 69117 Heidelberg, Germany), as well as Modeler's PIR format (Sali and Sanchez, 2000, Methods Mol. Biol. 143, 97-129).

In one embodiment of the present invention, alignment module 66 (FIG. 1) performs a pairwise alignment using an amino acid substitution matrix (e.g., alignment scoring table 68). An amino acid substitution matrix provides a numerical score for each of the possible pairings or substitutions that can be found at individual residue positions in an alignment. It will be appreciated that, in one embodiment, the amino acid substitution matrix is a (20×20) matrix, where elements of the matrix represent the score for substituting one of the naturally occurring amino acids with another of the naturally occurring amino acids. Furthermore, because there is no cost associated with conserving a residue (e.g. X→X), and substitutions of the class X→Y are identical to substitutions of the class Y→X, some amino acid substitution matrices could be represented by a (20×19×½) matrix. It will be appreciated however, that amino acid substitution matrices (e.g., alignment scoring tables 68) may provide information on the cost of other alignment parameters. Therefore, each amino acid substitution matrix 68 may, in fact, be larger than a (20×19×½) or a (20×20) matrix.

To illustrate how some alignment modules 66 in accordance with the present invention use an alignment scoring table 68, an example will be presented. If the residue at residue position i=11 in the target amino acid sequence 38 is A, and the residue at residue position i=11 in a query amino acid sequence 72 (FIG. 1) is Y, alignment scoring table 68 provides a numerical score for the substitution A Y. In this fashion, the score for each residue position in the target sequence is summed to determine the score for a particular pairwise alignment between the target sequence 38 and a query amino acid sequence 72.

In some embodiments of the present invention, a BLOSUM62 matrix is the amino acid substitution matrix 70 used by alignment module 66. The BLOSUM62 matrix is a derivative of the Dayhoff scoring matrix. The Dayhoff matrix provides a numerical value for substitution from any one of the twenty naturally occurring amino acids to another amino acid. (See Henikoff & Henikoff, 1993, Proteins 17, 49-61, 1993).

Another amino acid substitution matrix used in some embodiments of the present invention is the WAC matrix (Pac. Symp. Biocomput., 465-76, 1997). The WAC matrix is the result of a comprehensive analysis of the microenvironments surrounding the twenty naturally occurring amino acids. This analysis includes a comparison of amino acid environments with random control environments as well as with each of the other amino acid environments. These environments are described with a set of 21 features summarizing atomic, chemical group, residue, and secondary structural features. The environments are divided into radial shells of one Angstrom thickness to represent the distance of the features from the amino acid Cα atoms.

Still another amino acid substitution matrix used in accordance with some embodiments of the present invention is a Risler matrix (Risler et al., 1988, J. Mol. Biol. 204, 1019-29). In developing the Risler matrix, an amino acid a, in a protein PI is considered replaced by the amino acid a₂ in the structurally similar protein P2 when, after superposition of the two structures, the a₁ and a₂ C_{α} atoms are no more than 1.2 Angstroms apart. Using this criterion, amino acid pairs (substitutions) from various structures were analyzed by statistical methods to produce the Risler matrix.

Another preferred, non-limiting example of a alignment module 66 utilized for the comparison of sequences is the algorithm of Myers and Miller (Myers & Miller, CABIOS 4, 11-17, 1988). Such an algorithm is incorporated into the ALIGN program (version 2.0) which is part of the GCG sequence alignment software package. In one embodiment of the present invention, when utilizing the ALIGN program for comparing amino acid sequences, a PAM120 alignment scoring table 68 (Henikoff & Henikoff, 1992, Proc. Natl. Acad. Sci. USA, 89, 10915), a gap length penalty of 12, and a gap penalty of 4 is used. Additional algorithms for sequence analysis are known in the art and include ADVANCE and ADAM (Torellis & Robotti, 1994, Comput. Appl. Biosci., 10:3-5).

Many other amino acid substitution matrices are used in various embodiments of the present invention. Such tables include the PAM250 matrix (Henikoff & Henikoff, 1992, Proc. Natl. Acad. Sci. USA 89, p. 10915). Additional details on exemplary amino acid substitution matrices may be found at (http://sunflower.bio.indiana.edu/gcg-manual/afrc-gcg-doc/ch2-2.12.html; and Pearson, 1995, Protein Science 4, pp. 1145-1160).

In some embodiments of processing step 204, the sequence comparison problem is address in two parts: (1) pairwise alignment of query amino acid sequence 72 to target amino acid sequence 38 and (2) scoring the aligned amino acid sequences. In some embodiments this alignment involves a process of introducing “phases shifts” and “gaps” into one or both of the sequences being pairwise aligned in order to maximize the sequence similarity between two sequences. Scoring refers to the process of quantitatively expressing the relatedness of the aligned sequences.

### 6.7 Percent Identity and Percent Similarity

In some embodiments of the present invention, a query amino acid sequence 72 from a database of sequences 70 (FIG. 1) used in processing step 204 (FIG. 2A) is not added to the list of proteins used in a multiple sequence alignment 94 or an alignment profile unless the sequence shares a predetermined amount of sequence identity with the primary sequence of target amino acid sequence 38 (FIG. 2A). In such embodiments, the query sequence 72 has the requisite amount of sequence identity when at least 65%, at least 80%, or at least 90% of the residues in the second protein are identical to the residues in the first (target) protein. Sequence identity may be determined using an algorithm such as the BLAST algorithm, described in Altschul et al., 1990, J. Mol. Biol. 215, 403-410 as well as Karlin et al., 1993, PNAS USA 90, 5873-5787. A particularly useful BLAST program is the WU-BLAST-2 program. See Altschul et al., 1996, Methods in Enzymology 266, 460-480. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In some embodiments of the present invention, a query sequence 72 from a database of sequences 70 used in processing step 204 (FIG. 2A) is not added to the list of proteins used in a multiple sequence alignment 94 or an alignment profile unless the protein shares a predetermined amount of sequence similarity with the primary sequence of target amino acid sequence 38 (FIG. 1). In such embodiments, the query amino acid sequence 72 has the requisite amount of sequence similarity when at least 50%, at least 65%, at least 80%, or at least 90% of the residues in the query amino acid sequence 72 are similar (i.e. conservatively substituted) or identical to the residues in the target amino acid sequence 38. In one embodiment, percent similarity is defined as percent identity in addition to conservative substitutions (i.e. substitution with “similar amino acids”). Therefore, the definition for percent identity varies depending on which amino acid substitution matrix (e.g., alignment scoring table 68) is used. For a discussion of exemplary substitution matrices used in accordance with some embodiments of the present invention, see subsection 6.6, supra. In one embodiment of the present invention, “similar” amino acids are those with scores of >=0 in the substitution matrix.

### 6.8 Expectation Values

In some embodiments of the present invention, a query amino acid sequence 72 from a database amino acid sequence 70 used in processing step 204 (FIG. 2A) is not added to the list of sequences used in a multiple sequence alignment 94 or an alignment profile unless the second protein has an expectation value, with respect to the primary sequence of target amino acid sequence 38 (FIG. 1), that is within a predetermined range.

An expectation value is the number of distinct alignments with scores equivalent to or better than the one of interest, that are expected to occur in a database search purely by chance. The lower the E-value, the more significant the score. Thus, in some embodiments, the query amino acid sequence 72 must have an expectation value with respect to the target amino acid sequence 38 that is in a range of 1e⁻² to 1e⁻⁴⁰ for a given data base of sequences. An expectation value of 1e⁻² means that, for a given database, one sequence in a hundred would have an equivalent alignment score or better than the identified alignment. An expectation score of 1e⁻⁴⁰ means that, for a given database, one sequence in 10⁴⁰ would have an equivalent score or better than the identified alignment. In another embodiment of the invention, the query amino acid sequence 72 (FIG. 1) must have an expectation value with respect to the target amino acid sequence 38 that is less than 1e⁻¹⁰, for a given database of sequences. In another embodiment, the alignment between the target and query amino acid sequences must have an expectation value that is less than 1e⁻⁷, for a given database of sequences, in order to be incorporated into multiple sequence alignment 94.

### 6.9 Automated Form of Present Invention

### 6.9.1 General Algorithm

An automated form of the present invention will now be described with reference to FIG. 4. In one embodiment, a script, such as a perl script, is used to provide an automated version of the algorithm illustrated in FIGS. 2A and 2B. For more information on perl, see Wall & Schartz, 1991, Programming perl, O'Reilly & Associates, Sebastopol, Calif. In some embodiments, the script or module that is used to provide an automated version of the present invention is automation module 47 (FIG. 2). In step 402, a protein coordinate file is taken as input and the amino acid sequence of the coordinate file is obtained. It will be appreciated by those of skill in the art that there are many different methods for obtaining the target amino acid sequence that do not require a coordinate file for the target sequence. All such methods are within the scope of the present invention. In alternate embodiments, for example, step 402 comprises obtaining the target sequence from a sequence database or other electronic source.

In processing step 404, specific parameters that are used to regulate the search algorithm illustrated in FIGS. 2A and 2B are set to default values. In particular, the threshold distance (“cluster radius”) that is used in processing step 256 (FIG. 2B) is set to a default value on one embodiment of step 404. In one embodiment, the cluster radius is set to 5 Angstroms. In other embodiments, the cluster radius is set to a value of 3 Angstroms, 4 Angstroms, 6 Angstroms, 7 Angstroms, or some other value. Further, the e-value cut-off used in processing step 206 (“default expectation value”) is set in some embodiments of processing step 404. In one embodiment, the default expectation value is set to 1e⁻⁶. In other embodiments, the default expectation value is set to 1e⁻⁴, 1e⁻⁵, 1e⁻⁷, 1e⁻⁸, or some other value. In some embodiments, the threshold percentage that is used in processing step 244 is defined in step 404. The threshold percentage used in processing step 244 is used as a selection criterion. More specifically, in order for a residue at the i^{th} position of a target sequence to be considered a candidate active site sequence position, a threshold percentage of the aligned sequences in a multi-sequence alignment must include a residue at position i of any type. In some embodiments, this threshold percentage is set to one hundred percent. In some embodiments, a threshold percentage of “100 percent” requires that, for a given position i in the target sequence, there must be a residue (of any type) in each sequence in a multi-sequence alignment at the position that corresponds to target sequence position i. In more restrictive embodiments, a threshold percentage of “100 percent” requires that, for a given position i in the target sequence, there must be a residue of the same exact type in each sequence in a multi-sequence alignment at the position that corresponds to target sequence position i. In some embodiments the threshold percentage is set to 95 percent, 90 percent, 85 percent, 80 percent, or some other percentage.

In processing step 406, steps 204 through 258 (FIGS. 2A and 2B) are performed using the criteria set in processing step 404. In processing step 408, the success of processing steps 204 through 258 is queried. In particular, the question is asked whether a set of candidate active site sequence positions 55 (FIG. 1) were found. Recall that a list of candidate active site positions 53 (FIG. 1) is mapped to a three-dimensional representation of the target sequence in step 256 and that only those candidate active site positions that are within a threshold distance (cluster radius) of at least one other candidate active site sequence position are allowed into the set of candidate active site sequence positions 55 (FIG. 1). When a set of candidate active site sequence positions 55 is found (408-Yes), the process ends (460).

When steps 204 through 258 are unsuccessful (408-No), the default parameters set in processing step 404 are adjusted and steps 204 through 258 are rerun with the new parameter-set. The process of setting default parameters and running steps 204 through 258 continues until a set of candidate active site sequence positions 55 is found. What is illustrated in FIG. 4 is one algorithm for adjusting the default parameters (steps 420 through 442). It will be appreciated that there are many other algorithms for adjusting the default parameters other than those disclosed and FIG. 4, and all such algorithms are within the scope of the present invention. Specifically, the present invention encompasses all algorithms for setting default search parameters, running steps 202 through 258 of FIG. 2, resetting one or more default search parameters, and rerunning steps 204 through 258 of FIG. 2 until a set of candidate active sequence position 55 is found.

In processing step 420, the cut-off radius increased by one angstrom. In processing step 422, the question is asked whether a maximum threshold of eight Angstroms has been exceeded. If not (422-No), steps 204 through 258 are performed with the new cluster radius. Of course, in this instance, only processing step 256 needs to be rerun since this is the only step in which the cluster radius is applied. If a set of candidate active site sequence positions 55 are still not found with the relaxed cluster radius (408-No), step 420 will further increase the cluster radius and steps 204 through 258 (or just step 258) will be repeated until the cluster radius exceeds eight angstroms (422-Yes). Those of skill in the art will appreciate that the upper cluster radius threshold may some value other than eight Angstroms (e.g., 5, 6, 6.5, 7, 7.5, 8.5, 9, or 10 Angstroms) and all such values are within the scope of the present invention.

When the cluster radius exceeds eight Angstroms, or some other defined upper cluster threshold length (422-Yes), process control passes to step 430, where the question is asked whether the e-value cut-off has already been set to 1e⁻¹². If not (430-No), the e-value cut-off is in fact set to 1e⁻¹² and the cluster radius is reset to five Angstroms (step 432). Then, process control passes to step 406, where steps 204 through 248 (FIGS. 2A and 2B) are rerun with the new default parameters. In particular, in processing step 206, the subset of aligned query sequences is chosen such that each aligned query sequence in the subset has an overall sequence similarity or identity with the target sequence that is less than the expectation value 1e⁻¹². That means that, in order to be selected for the subset of aligned query sequences in step 206, a query sequence 72 (FIG. 1) must have an expectation value of 1e⁻¹² or less. This is a more stringent requirement than 1e⁻⁶, and it will result in the creation of a more homologous subset of aligned sequences in step 206. Expectation values other than 1e⁻¹² may be used in step 432, for example, the expectation value could be set to 1e⁻⁹, 1e⁻¹³, 1e⁻¹⁴, 1e⁻¹⁵, or 1e⁻¹⁶. The only requirement for processing step 432 is that the e-value cut-off is set to a more stringent value so that the sequences selected in step 206, on average, share a greater sequence homology with the target sequence. Steps 406 through 422 are repeated with the more stringent e-value cutoff until either (i) a set of candidate active site sequence positions 55 are found (408-Yes) or (ii) the cluster radius exceeds a maximum threshold value (422-Yes).

When the cluster radius exceeds a maximum threshold (422-Yes) and the e-value has been set to a more stringent value (430-Yes), the threshold percentage parameter is relaxed by an amount. As illustrated in FIG. 4, the threshold percentage parameter is relaxed by five percent (step 440). Further, the cluster radius is set to five Angstroms. Then, steps 204 through 258 are repeated (step 406) with the new parameters settings. In particular, relaxation of the threshold identity parameter affects step 244 because a complete match at a given residue position i is no longer required. Thus, relaxation of the threshold identity parameter has the effect of increasing the set of candidate active site sequence positions 55 that will be considered in step 256. Processing steps 406 through 442 are repeated in the manner shown in FIG. 4 until a set of candidate active site sequence positions 55 is found.

### 6.9.2 Specific Embodiment

In one embodiment, each of the following steps is repeated until a potential cluster of functional residues is found or the limiting condition is reached. If a limiting condition is reached, the next set of default parameters is tested until the limiting condition is reached.

1. The cluster radius is increased stepwise by 1 Å. Limiting condition: cluster radius of 8 Å.

2. The e-value cut-off is decreased to 1e⁻¹², the cluster radius is set to 5 Å. The cluster radius is relaxed (increased) in 1 Å steps. Limiting condition: cluster radius of 8 Å.

3. The e-value is set to 1e⁻¹² and the cluster radius is set to 5 Å. The identity is relaxed stepwise by five percent or some other step percentage, such as three percent, four percent or eight percent. For each value of percent identity tested, the cluster radius is relaxed by 1 Å up to a maximum of 8 Å. This step is repeated until a cluster is found.

Some embodiments of the present invention make an educated guess about the residues that might play a role in binding the substrate/cofactor. In such embodiments, the output from embodiments of the invention such as that disclosed in Figure FIG. 2 or FIG. 4 is accepted as the input. For each of the proposed clusters of functionally important residues identified by the processes of FIG. 2 and/or FIG. 4 (e.g., set of candidate active site sequence positions), a set of binding site residues is identified. The criteria used for annotating a residue as a potential binding site residue are as follows:

1. The residue has to be at least eighty percent conserved in the family of similar sequences identified by the blast search during the initial run (step 204 through 258 of FIG. 2).

2. The residue has to be within 4 Å of at least one of the residues identified as of catalytic importance.

### 7. EXAMPLES

Exemplary systems and methods in accordance with the present invention have been described. Table 8 summarizes results for five proteins (1CUD, 1HD1, 4LIP, 1ALH, and 1RSC) chosen from three-dimensional representations 44 found in the Protein Data Bank (http://www.rcsb.org/pdb). Each structure in the Protein Data Bank represents a particular macromolecule, such as a protein, and is given a unique four letter accession number (e.g. 1CUD).

The exemplary systems were chosen essentially randomly from a subset of proteins for which the active site information is available and listed in the IMB Jena image library site database (http://www.imbjena.de/ImgLibPDB/pages/siteDir/IMAGE_SITE.shtml). The nomenclature used to identify positions in proteins in the experimental data is amino acid code followed by position (e.g. S120, for one letter code, or Ser120, for three letter code).

TABLE 8
Comparison of predicted and actual active site residues
for five target sequences 38 examined using the methods
of the present invention

| | | Actual active site | |
|---|---|---|---|
| PDB | | residues (source: IMB | Predicted active site |
| code | Protein | Jena) | residues |
| 1CUD | Cutinase | S120, D175, H188 | All three residues |
| | | | occur in the largest |
| | | | cluster identified |
| | | | by the methods of |
| | | | the present invention |
| 1HDI | Phospho- | D23, R38, H62, R65, | D23 and R38 occur |
| | glycerate | R122, R170 | in the cluster while |
| | kinase | | R170 is beyond a |
| | | | five angstrom distance |
| | | | cut-off from the cluster |
| 4LIP | Lipase | S87, D264, H286 | All three residues |
| | | | occur in the largest |
| | | | cluster identified |
| | | | by the present |
| | | | invention |
| 1ALH | Alkaline | D51, S102, D153, | The only cluster |
| | phosphatase | T155, R166, E322, | predicted by the |
| | | D327, K328, H331, | present invention has |
| | | H370, H412 | D51, T155, E322, |
| | | | D327, H331, H370 |
| 1RSC | RUBISCO | K175, D203, E204, | D203, E204, H294 and |
| | | H294, K334, S379 | S379 are present in the |
| | | | largest predicted |
| | | | cluster |

In the case of 1HD1, 1ALH and 1RSC, due to the internal inconsistencies of the PDB files, the residue numberings in the Jena records (column 3 in Table 8) and in the actual output of one embodiment of the methods of the present invention differ from each other by three. This is taken into account before reporting the results in 10 columns three and four in Table 8. In each of the examples presented below, the amino acid sequence database 70 that was used as a source of query sequences was Genbank (http://www.ncbi.nlm.nih.gov/Genbank/). More detailed results are now presented for each of the proteins listed in Table 8.

### 7.1. 1CUD

1CUD (Longhi et al., 1996, Proteins 26, 442). A total of 16 amino acid sequences having an expectation value less than 1e−06 were identified for target sequence 1CUD. This pairwise alignment and a threshold distance of 5.1 angstroms were used. Results of calculations made in accordance with the present invention are summarized in Table 9.

TABLE 9
Results for 1CUD

| Residue | | conserved | subst . . . | . . . times . . . to | Contacts |
|---|---|---|---|---|---|
| 31 | C | 16 | 0 | | A31: 2 contacts with A28 A109 |
| 40 | R | 16 | 0 | | A40: 1 contacts with A78 |
| 109 | C | 16 | 0 | | A109: 1 contacts with A31 |
| 120 | S | 16 | 0 | | A120: 2 contacts with A188 |
| 171 | C | 16 | 0 | | A171: 1 contacts with A178 |
| 178 | C | 16 | 0 | | A178: 3 contacts with A171 175 |
| 188 | H | 15 | 0 | | A188: 3 contacts with A120 A175 |
| 13 | R | 8 | 0 | | A13: 0 contacts |
| 3 | T | 3 | 0 | | A3: 0 contacts |
| 4 | S | 3 | 0 | | A4: 0 contacts |
| 211 | R | 3 | 0 | | A211: 3 contacts with A33 A113 |
| 213 | S | 3 | 0 | | A213 contacts |
| 44 | E | 15 | 1 | 1Q | A44: 1 contacts with A50 |
| 175 | D | 15 | 1 | 1H | A175: 3 contacts with A178 A188 |
| 11 | E | 7 | 1 | 1Q | A11: 0 contacts |
| 9 | E | 4 | 1 | 1D | A9: 0 contacts |
| 208 | R | 4 | 1 | 1G | A208: 0 contacts |
| 167 | T | 12 | 4 | 3V 1L | A167: 0 contacts |
| 42 | S | 9 | 7 | 6T 1R | A42: 0 contacts |
| 194 | D | 9 | 5 | 3E 2G | A194: 1 contacts with A172 |
| 165 | D | 6 | 10 | 8S 2E | A165: 0 contacts |
| 166 | R | 6 | 10 | 9K 1D | A166: 0 contacts |
| 18 | T | 6 | 4 | 3S 1I | A18: 3 contacts with A17 A19 |
| 134 | D | 4 | 12 | 8P 4S | A134: 0 contacts |
| 22 | D | 4 | 6 | 5E 1- | A22: 3 contacts with A20 |
| 206 | K | 4 | 6 | 4R 2Q | A206: 0 contacts |

The methods of the present invention predicted that the clusters most likely to participate in catalysis were: (CYS31, CYS109) and (SER120, CYS171, ASP175, CYS178, HIS188). The actual active site residues are SER120, ASP175, and HIS188.

### 7.2. 1HD1

1HD1 (Szilagyi et al, 2001, J. Mol. Biol., 306, 499) A total of 229 amino acid sequences having an expectation value less than 1e−06 were identified for target sequence 1HD1. This pairwise alignment and a threshold distance of 5.1 angstroms were used. Results of calculations made in accordance with the present invention are summarized in Table 10.

TABLE 10
Results for 1HDI

| Residue | | conserved | subst . . . | . . . times . . . to | Contacts |
|---|---|---|---|---|---|
| 167 | R | 179 | 0 | | A167: A62 A119 A166 A169 |
| 340 | E | 179 | 0 | | A340: A371 |
| 389 | S | 179 | 0 | | A389: A189 A387 A390 A395 |
| 311 | D | 178 | 0 | | A311: A312 A348 |
| 20 | D | 167 | 0 | | A20: A35 A58 A59 A164 |
| 2 | K | 87 | 0 | | A2: A408 |
| 281 | D | 184 | 2 | 2X | A281: A316 |
| 166 | H | 178 | 1 | 1Q | A166: A167 A397 |
| 119 | R | 177 | 1 | 1C | A119: A59 A62 A125 A167 A169 |
| 160 | D | 176 | 1 | 1E | A160: A42 |
| 35 | R | 167 | 2 | 2K | A35: A20 |
| 348 | T | 161 | 19 | 19S | A348: A311 |
| 18 | R | 161 | 1 | 1H | A18: A58 A59 A124 A164 A171 |
| 390 | T | 175 | 4 | 3S 1I | A390: A189 A389 |
| 116 | E | 172 | 6 | 3Q 3D | A116: A73 |
| 59 | H | 171 | 5 | 4Y 1- | A59: A18 A20 A58 A119 A164 |
| 371 | D | 161 | 19 | 13H 6E | A371: A340 A374 |
| 42 | S | 29 | 142 | 135T 7A | A42: A160 |

The methods of the present invention predicted that the clusters most likely to participate in catalysis were (ASP20, ARG35), (ASP311, THR348) or (GLU340, ASP371). The actual active site residues are ASP23, ARG38, HIS62, ARG65, ARG122, and ARG170. In Table 10, due to the internal inconsistencies of the PDB files, the residue numberings in the Jena records (column 3 in Table 8) and in the actual output of one embodiment of the methods of the present invention differ from each other by three.

### 7.3. 4LIP

4LIP (http://www.rcsb.org/pdb/under accession ID 4LIP). A total of 33 amino acid sequences having an expectation value less than 1e−06 were identified for target sequence 4LIP. This pairwise alignment and a threshold distance of 5.1 angstroms were used. Results of calculations made in accordance with the present invention are summarized in Table 11.

TABLE 11
Results for 4LIP

| Residue | | conserved | subst . . . | . . . times . . . to | Contacts |
|---|---|---|---|---|---|
| 87 | S | 31 | 0 | | D87: D86 D286 |
| 94 | R | 31 | 0 | | D94: D114 |
| 106 | S | 29 | 0 | | D106: D108 |
| 121 | D | 27 | 0 | | D121: D168 D169 D258 |
| 208 | S | 24 | 0 | | D208: D109 |
| 264 | D | 24 | 0 | | D264: D286 D289 |
| 271 | S | 24 | 0 | | D271: D268 |
| 286 | H | 24 | 0 | | D286: D86 D87 D264 D289 |
| 288 | D | 24 | 0 | | D288: D230 D242 |
| 314 | R | 21 | 0 | | D314: D311 |
| 86 | H | 30 | 1 | 1Y | D86: D87 D286 D289 |
| 108 | T | 28 | 1 | 1S | D108: D106 D311 |
| 242 | D | 21 | 3 | 3- | D242: D244 D245 D288 |
| 311 | H | 21 | 1 | 1Q | D311: D108 D314 |
| 159 | D | 15 | 9 | 9N | D159: 0 contacts |
| 15 | H | 30 | 3 | 2P 1N | D15: D61 |
| 7 | T | 29 | 3 | 2P 1H | D7: 0 contacts |
| 117 | S | 26 | 3 | 2T 1A | D117: D118 |
| 63 | E | 25 | 6 | 3A 3D | D63: 0 contacts |
| 169 | T | 18 | 6 | 5S 1R | D169: D121 |
| 316 | K | 17 | 4 | 2Q 2R | D316: 0 contacts |
| 109 | T | 11 | 18 | 17S 1A | D109: D112 D114 D208 |
| 228 | D | 9 | 15 | 13- 2L | D228: D230 D231 |
| 166 | T | 8 | 16 | 11S 5A | D166: D54 |
| 224 | T | 8 | 16 | 13- 3K | D224: D219 |
| 280 | T | 7 | 17 | 14D 3S | D280: D269 D281 |

The methods of the present invention predicted that the clusters most likely to participate in catalysis were (SER87, ASP264, HIS286) or (SER106, THR108). The actual active site residues are SER87, ASP264, and HIS286.

### 7.4. 1ALH

1ALH (Tibbitts et al., 1994, Protein Science 3, 2005). A total of 118 amino acid sequences having an expectation value less than 1e−06 were identified for target sequence 1ALH. This pairwise alignment and a threshold distance of 5.1 angstroms were used. Results of calculations made in accordance with the present invention are summarized in Table 12.

TABLE 12
Results for 1ALH

| Residue | | conserved | subst . . . | . . . times . . . to | Contacts |
|---|---|---|---|---|---|
| 48 | D | 114 | 0 | | A48: A99 A150 A152 A319 A324 A325 A367 |
| 319 | E | 108 | 0 | | A319: A48 A144 A152 A325 |
| 324 | D | 107 | 0 | | A324: A48 A99 A325 A328 A367 A369 A409 |
| 328 | H | 107 | 0 | | A328: A324 A369 A406 A409 |
| 367 | H | 107 | 0 | | A367: A48 A99 A102 A324 |
| 99 | S | 107 | 1 | 1M | A99: A48 A163 A324 A367 A409 |
| 111 | K | 107 | 1 | 1R | A111: A431 |
| 338 | E | 95 | 12 | 12D | A338: A145 A322 A341 |
| 152 | T | 69 | 40 | 40S | A152: A48 A319 |
| 409 | H | 53 | 1 | 1D | A409: A99 A324 A328 A367 A369 |
| 7 | R | 42 | 1 | 1Q | A7: 0 contacts |
| 150 | D | 32 | 77 | 77H | A150: A48 A147 A163 A325 |
| 145 | T | 107 | 2 | 1H 1S | A145: A144 A322 A338 |
| 163 | R | 106 | 3 | 2-1S | A163: A98 A99 A150 |
| 98 | D | 103 | 5 | 3E 2S | A98: A97 A163 |
| 40 | K | 100 | 13 | 12R 1A | A40: A356 |
| 359 | T | 100 | 7 | 5S 2A | A359: A356 |
| 433 | T | 41 | 11 | 6N 5S | A433: A431 A434 |
| 431 | D | 21 | 32 | 30E 2N | A431: A82 A111 A433 A434 |
| 446 | K | 15 | 4 | 3R 1Q | A446: 0 contacts |

The methods of the present invention predicted that the clusters most likely to participate in catalysis were (ASP48, ASP150, THR152, GLU319, ASP324, HIS328, HIS367). The actual active site residues are ASP51, SER102, ASP153, THR155, ARG166, GLU322, ASP327, LYS328, HIS331, HIS370, and HIS412. In Table 12, due to the internal inconsistencies of the PDB files, the residue numberings in the Jena records (column 3 in Table 8) and in the actual output of one embodiment of the methods of the present invention differ from each other by three.

### 7.5. 1RSC

1RSC (Newman, 1994, Structure 2, 495). A total of 248 amino acid sequences having an expectation value less than 1e−06 were identified for target sequence 1RSC. This pairwise alignment and a threshold distance of 5.1 angstroms were used. Results of calculations made in accordance with the present invention are summarized in Table 13.

TABLE 13.1
Results for 1RSC

| Residue | conserved | subst . . . | . . . times . . . to | Contacts |
|---|---|---|---|---|
| 174 K | 248 | 0 | | A174: A172 A200 A201 |
| 178 S | 248 | 0 | | A178: 0 contacts |
| 180 K | 248 | 0 | | A180: A184 |
| 184 R | 248 | 0 | | A184: A180 |
| 188 E | 248 | 0 | | A188: A191 |
| 189 C | 248 | 0 | | A189: A169 |
| 191 R | 248 | 0 | | A191: A188 A228 |
| 195 D | 248 | 0 | | A195: A161 A164 A231 A418 |
| 197 T | 248 | 0 | | A197: A235 |
| 198 K | 248 | 0 | | A198: A170 A200 A201 A289 A291 A324 |
| 199 D | 248 | 0 | | A199: A214 A235 |
| 200 D | 248 | 0 | | A200: A170 A172 A174 A198 A201 A291 |
| 201 E | 248 | 0 | | A201: A174 A198 A200 A291 |
| 210 R | 248 | 0 | | A210: A205 |
| 212 R | 248 | 0 | | A212: A256 |
| 213 D | 248 | 0 | | A213: 0 contacts |
| 224 K | 248 | 0 | | A224: A228 |
| 228 E | 248 | 0 | | A228: A191 A224 |
| 229 T | 248 | 0 | | A229: A231 A233 |
| 231 E | 248 | 0 | | A231: A161 A164 A195 A229 A233 A418 |
| 233 K | 248 | 0 | | A233: A229 A231 A418 |
| 235 H | 248 | 0 | | A235: A197 A199 |
| 240 T | 248 | 0 | | A240: A264 A265 |
| 243 T | 248 | 0 | | A243: A245 |
| 245 E | 248 | 0 | | A245: A243 A244 |
| 249 K | 248 | 0 | | A249: A246 |
| 250 R | 248 | 0 | | A250: A205 A246 |
| 264 H | 248 | 0 | | A264: A240 |
| 265 D | 248 | 0 | | A265: A240 A268 A291 |
| 268 T | 248 | 0 | | A268: 2 contacts with A265 |
| 272 T | 248 | 0 | | A272: A111 |
| 281 C | 248 | 0 | | A281: 0 contacts |
| 282 R | 248 | 0 | | A282: 0 contacts |
| 283 D | 248 | 0 | | A283: 0 contacts |
| 289 H | 248 | 0 | | A289: A198 A322 A324 |
| 291 H | 248 | 0 | | A291: A198 A200 A201 A265 A324 |
| 292 R | 248 | 0 | | A292: A295 A299 |
| 299 D | 248 | 0 | | A299: A292 A295 |
| 300 R | 248 | 0 | | A300: A333 |
| 304 H | 248 | 0 | | A304: 0 contacts |
| 309 R | 248 | 0 | | A309: A133 A134 A307 A338 |
| 316 R | 248 | 0 | | A316: 0 contacts |
| 318 S | 248 | 0 | | A318: A275 |
| 322 H | 248 | 0 | | A322: A155 A289 |
| 324 H | 248 | 0 | | A324: A198 A289 A291 A376 |
| 327 T | 248 | 0 | | A327: 0 contacts |
| 331 K | 248 | 0 | | A331: 0 contacts |

TABLE 13.2
Results for IRSC

| Residue | | conserved | subst . . . | . . . times . . . to | Contacts |
|---|---|---|---|---|---|
| 333 | E | 248 | 0 | | A333: A300 A468 |
| 347 | R | 248 | 0 | | A347: A344 A348 |
| 354 | D | 248 | 0 | | A354: A356 A357 |
| 357 | R | 248 | 0 | | A357: A344 A354 |
| 364 | D | 248 | 0 | | A364: A349 A350 |
| 367 | S | 248 | 0 | | A367: A349 |
| 376 | S | 248 | 0 | | A376: A170 A324 |
| 380 | H | 248 | 0 | | A380: A383 |
| 383 | H | 248 | 0 | | A383: A380 |
| 393 | D | 248 | 0 | | A393: A428 |
| 394 | D | 248 | 0 | | A394: A156 |
| 403 | T | 248 | 0 | | A403: A169 |
| 406 | H | 248 | 0 | | A406: 0 contacts |
| 418 | R | 248 | 0 | | A418: A164 A195 A231 A233 A422 |
| 422 | E | 248 | 0 | | A422: A164 A418 |
| 424 | C | 248 | 0 | | A424: 0 contacts |
| 428 | R | 248 | 0 | | A428: A393 |
| 430 | E | 248 | 0 | | A430: A432 |
| 432 | R | 248 | 0 | | A432: A430 A437 A444 |
| 449 | S | 248 | 0 | | A449: A451 |
| 451 | E | 248 | 0 | | A451: A449 |
| 460 | K | 248 | 0 | | A460: 0 contacts |
| 470 | D | 243 | 0 | | A470: A131 A307 A338 |
| 4 | T | 105 | 0 | | A4: 0 contacts |
| 20 | T | 247 | 1 | 1N | A20: A15 |
| 38 | R | 247 | 1 | 1P | A38: A131 A133 A302 |
| 64 | T | 247 | 1 | 1R | A64: 0 contacts |
| 90 | E | 247 | 1 | 1D | A90: A302 |
| 111 | T | 247 | 1 | 1A | A111: A109 A272 |
| 116 | S | 247 | 1 | 1Y | A116: 0 contacts |
| 128 | R | 247 | 1 | 1K | A128: 0 contacts |
| 205 | S | 247 | 1 | 1X | A205: A210 A250 |
| 214 | R | 247 | 1 | 1C | A214: A199 |
| 275 | T | 247 | 1 | 1N | A275: A318 |
| 295 | H | 247 | 1 | 1Q | A295: A292 A299 |
| 313 | K | 247 | 1 | 1X | A313: A134 |
| 321 | D | 247 | 1 | 1E | A321: A150 |
| 344 | D | 247 | 1 | 1V | A344: A347 A348 A357 |
| 389 | E | 247 | 1 | 1D | A389: A433 |
| 443 | R | 247 | 1 | 1X | A443: A440 |
| 463 | K | 247 | 1 | 1N | A463: A461 |
| 133 | E | 246 | 2 | 2D | A133: A38 A131 A309 |
| 256 | E | 246 | 2 | 2D | A256: 1 contacts with A212 |
| 355 | R | 246 | 2 | 2K | A355: A85 |
| 29 | K | 245 | 3 | 3A | A29: 0 contacts |
| 349 | D | 245 | 3 | 3N | A349: A364 A367 |
| 15 | K | 243 | 5 | 5Q | A15: A11 A20 A65 |
| 16 | D | 238 | 10 | 10E | A16: A18 A49 |

TABLE 13.3
Results for 1RSC

| | | con- | | | |
|---|---|---|---|---|---|
| Residue | | served | subst . . . | . . . times . . . to | Contacts |
| 246 | E | 235 | 13 | 13D | A246: A249 |
| | | | | | A250 |
| 276 | T | 63 | 185 | 185S | A276: 0 contacts |
| 225 | S | 43 | 205 | 205A | A225: 0 contacts |
| 40 | S | 17 | 231 | 231T | A40: A93 A129 |
| 255 | K | 11 | 237 | 237R | A255: 0 contacts |
| 335 | D | 6 | 242 | 242E | A335: A338 |
| | | | | | A468 |
| 336 | K | 4 | 244 | 244R | A336: 0 contacts |
| 48 | D | 2 | 246 | 246E | A48: A18 |
| 93 | S | 2 | 246 | 246Q | A93: A40 |
| 129 | S | 2 | 246 | 246A | A129: A40 |
| 339 | T | 246 | 2 | 1X 1A | A339: A338 |
| 244 | C | 245 | 3 | 2N 1A | A244: A245 |
| 307 | H | 245 | 3 | 2P 1T | A307: A131 |
| | | | | | A309 A338 A470 |
| 447 | K | 245 | 3 | 2R 1X | A447: 0 contacts |
| 65 | T | 244 | 4 | 3S 1A | A65: A11 A15 |
| 395 | S | 244 | 4 | 3A 1F | A395: 0 contacts |
| 437 | E | 244 | 4 | 3Q 1A | A437: A432 |
| | | | | | A436 |
| 11 | K | 243 | 5 | 3Q 2N | A11: A15 A65 |
| 461 | E | 241 | 7 | 6A 1Q | A461: A463 |
| 436 | R | 240 | 8 | 7S 1T | A436: A433 |
| | | | | | A437 |
| 91 | E | 237 | 11 | 10D 1K | A91: 0 contacts |
| 348 | E | 9 | 239 | 238D 1I | A348: A344 |
| | | | | | A347 |
| 415 | T | 9 | 239 | 238V 1A | A415: 0 contacts |
| 302 | R | 8 | 240 | 239K 1X | A302: A38 A90 |
| 220 | D | 5 | 243 | 242E 1G | A220: 0 contacts |
| 6 | S | 4 | 114 | 103A 11T | A6: 0 contacts |
| 80 | K | 2 | 246 | 243R 3X | A80: 0 contacts |

The methods of the present invention predicted that the clusters most likely to participate in catalysis were (ASP16, GLU49), (THR31, ASP32), (GLU57, THR62), (SER58, SER59, THR60), (THR68, ASP69),(ASP75, LYS78), (ARG76, ASP103), (GLU85, ARG355), (GLU106, SER109), (ARG131, GLU133, ASP134, ARG309, ASP470), (HIS150, ASP321), (GLU155, THR170, LYS172, LYS174, LYS198, ASP200, GLU201, THR240, HIS264, ASP265, THR268, HIS289, HIS291, HIS322, HIS324, SER376), (ARG156, ASP394),(LYS161, ARG164, ASP195, THR229, GLU231, LYS233, ARG418, GLU422), (CYS169, CYS189, THR403), (LYS180, ARG184), (GLU188, ARG191, LYS224, GLU228), (THR197, ASP199, HIS235), (ARG212, GLU256), (THR243, GLU245), (GLU246, LYS249, ARG250), (ARG292, ASP299), (ARG300, GLU333), (ASP354, ARG357), (HIS380, HIS383), (ASP393, ARG428), (GLU430, ARG432), and (SER449, GLU451). The actual active site residues are LYS175, ASP203, GLU204, HIS294, LYS334, and SER379. In Table 13, due to the internal inconsistencies of the PDB files, the residue numberings in the Jena records (column 3 in Table 8) and in the actual output of one embodiment of the methods of the present invention differ from each other by three.

### 8.0. REFERENCES CITED

All references cited herein are incorporated herein by reference in their entirety and for all purposes to the same extent as if each individual publication or patent or patent application was specifically and individually indicated to be incorporated by reference in its entirety for all purposes.

### 9.0 CONCLUSION

The present invention can be implemented as a computer program product that comprises a computer program mechanism embedded in a computer readable storage medium. For instance, the computer program product could contain modules shown in FIG. 1. These program modules may be stored on a CD-ROM, magnetic disk storage product, or any other computer readable data or program storage product. The software modules in the computer program product may also be distributed electronically, via the Internet or otherwise, by transmission of a computer data signal (in which software modules are embedded) on a carrier wave.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

I claim:

1. A method for selecting a plurality of candidate active site positions in a target sequence, the method comprising:
aligning each query sequence in a plurality of query sequences to said target sequence to form a set of aligned query sequences;
choosing a subset of aligned query sequences from said set of aligned query sequences, each aligned query sequence in said subset of aligned query sequences satisfying a predetermined criterion;
identifying substitution types for each residue position i in said target sequence, each substitution type characterized by a difference between (i) a residue identity at a residue position i in an aligned query sequence in said subset of aligned query sequences and (ii) said residue identity at said residue position i in said target sequence; and
selecting said plurality of candidate active site positions, each candidate active site position in said plurality of candidate active site positions identified as a residue position i in said target sequence in which (i) a residue identity at said residue position i in said target sequence is in an allowed class of amino acids, (ii) each substitution type identified for said residue position i in said identifying step is in an allowed class of substitution types, and (iii) a threshold percentage of said subset of aligned query sequences include a residue that is aligned with said residue position i in said target sequence.

2. The method of claim 1, further comprising the step of obtaining a set of candidate active site positions from said plurality of candidate active site positions, each candidate active site position in said set of candidate active site positions having a property that, when a first residue corresponding to said candidate active site position is mapped to a three-dimensional representation of said target sequence, a polar or charged atom of a side-chain of said first residue is within a threshold distance of at least one other polar or charged atom belonging to a side-chain of a second residue, wherein said second residue corresponds to a second candidate active site position in said set of candidate active site positions that has been mapped to said three-dimensional representation.

3. The method of claim 1 wherein, for each query sequence in said plurality of query sequences, said aligning step further comprises the steps of:
positioning said query sequence and said target sequence relative to each other;
scoring a sequence similarity between said query sequence and said target sequence after said positioning step; and
repeating said positioning step and said scoring step until said sequence similarity between said query sequence and said target sequence has been maximized or said repeating step has been executed a predetermined number of times.

4. The method of claim 3 wherein said positioning step includes an introduction of a “phase shift” into said query sequence or said target sequence.

5. The method of claim 3 wherein said positioning step includes an introduction of a “gap” into said query sequence or said target sequence.

6. The method of claim 5 wherein said positioning step includes extending said “gap” into said query sequence or said target sequence.

7. The method of claim 1 wherein said aligning step invokes a global comparison method or a local comparison method.

8. The method of claim 1 wherein said aligning step comprises pairwise alignment of each query sequence in said plurality of query sequences to said target sequence using BLAST, PSI-BLAST, WU-BLAST-2, MEGABLAST, or FASTA; wherein an alignment scoring table used for scoring said sequence similarity between each query sequence in said plurality of query sequences to said target sequence is selected from the group consisting of Blosum62, a Dayhoff table, PAM250, a WAC matrix, a Risler matrix, and PAM120.

9. The method of claim 8 wherein said predetermined criterion is an overall sequence similarity with said target sequence that is less than an expectation value selected from a range of about 1e−2 to about 1e−9.

10. The method of claim 9 wherein said expectation value is about 1e−6.

11. The method of claim 1 wherein said allowed class of amino acids consists of R, K, H, D, E, S, T, and C.

12. The method of claim 1 wherein said allowed class of substitution types consists of R→K, K→R, K→H, H→K, H→R, R→H, D→E, E→D, E→H, H→E, D→H, H→D, S→T, and T→S.

13. The method of claim 1 wherein said threshold percentage of said set of aligned query sequences comprises about 80 percent of said set of aligned query sequences.

14. The method of claim 2 wherein said three-dimensional representation of said target sequence is selected from the group consisting of a crystal structure of said target sequence, a structure of said target sequence derived by nuclear magnetic resonance, and a homology model of said target sequence.

15. The method of claim 1 wherein said polar atom is selected from the group consisting of OD1, OD2, OE1, OE2, NZ, NE, NH1, NH2, ND1, NE2, SG, OH, OG, OG1, ND2, and NE2.

16. The method of claim 2 wherein said threshold distance is selected from the range of about 2.0 Å to about 7.0 Å.

17. The method of claim 16 wherein said threshold distance is selected from the range of about 3.0 Å to about 6.0 Å.

18. The method of claim 2, the method further comprising:
adjusting one or more of said predetermined criterion, said threshold percentage, and said threshold distance; and
repeating said choosing, said identifying, said selecting, and said obtaining.

19. The method of claim 2, the method further comprising:
adjusting said threshold distance; and
repeating said obtaining.

20. The method of claim 2, the method further comprising:
setting said predetermined criterion, said threshold percentage, and said threshold distance;
performing said choosing, said identifying, said selecting, and said obtaining;
adjusting one or more of said predetermined criterion, said threshold percentage, and said threshold distance; and
repeating said choosing, said identifying, said selecting, and said obtaining.

21. A computer program product for use in conjunction with a computer having a processor, said computer program product comprising a computer readable storage medium and a computer program mechanism embedded therein, the computer program mechanism for selecting a plurality of candidate active site positions in a target sequence, the computer program mechanism causing the processor to execute the steps of:
aligning a plurality of query sequences to said target sequence to form a set of aligned query sequences;
choosing a subset of aligned query sequences from said set of aligned query sequences, each aligned query sequence in said subset of aligned query sequences satisfying a predetermined criterion;
identifying substitution types for each residue position i in said target sequence, each substitution type characterized by a difference between (i) a residue identity at a residue position i in an aligned query sequence in said set of aligned query sequences and (ii) said residue identity at said residue position i in said target sequence; and
selecting a plurality of candidate active site positions, each candidate active site position in said plurality of candidate active site positions identified as a residue position i in said target sequence in which (i) a residue identity at said residue position i in said target sequence is in an allowed class of amino acids, (ii) each substitution type identified for said residue position i in said target sequence is in an allowed class of substitution types, and (iii) a threshold percentage of said set of aligned query sequences includes a residue that is aligned with said residue position i in said target sequence.

22. The computer program product of claim 21, the computer program mechanism causing the processor to execute the additional step of:
obtaining a set of candidate active site positions from said plurality of candidate active site positions, each candidate active site position in said set of candidate active site positions having a property that, when a first residue corresponding to said candidate active site position is mapped to a three-dimensional representation of said target sequence that corresponds to said position, a polar or charged atom of a side-chain of said first residue is within a threshold distance of at least one other polar or charged atom belonging to a side-chain of a second residue, wherein said second residue corresponds to a second candidate active site position in said set of candidate active site positions that has been mapped to said three-dimensional representation.

23. The computer program product of claim 21 wherein, for each query sequence in said plurality of query sequences, said aligning step further comprises the steps of:
positioning said query sequence and said target sequence relative to each other;
scoring a sequence similarity between said amino acid sequence and said target sequence after said positioning step; and
repeating said positioning step and said scoring step until said sequence similarity between said query sequence and said target sequence has been maximized or said repeating step has been executed a predetermined number of times.

24. The computer program product of claim 23 wherein said positioning step includes an introduction of a “phase shift” into said query sequence or said target sequence.

25. The computer program product of claim 23 wherein said positioning step includes an introduction of a “gap” into said query sequence or said target sequence.

26. The computer program product of claim 25 wherein said positioning step includes extending said “gap” in said query sequence or said target sequence.

27. The computer program product of claim 21 wherein said aligning step invokes a global comparison method or a local comparison method.

28. The computer program product of claim 21 wherein said aligning step comprises pairwise alignment of each query sequence in said plurality of query sequences to said target sequence using BLAST, PSI-BLAST, WU-BLAST-2, MEGABLAST, or FASTA; wherein an alignment scoring table used for scoring said sequence similarity between each query sequence in said plurality of query sequences to said target sequence is selected from the group consisting of Blosum62, a Dayhoff table, PAM250, a WAC matrix, a Risler matrix, and PAM120.

29. The computer program product of claim 28 wherein said predetermined criterion is an overall sequence similarity with said target sequence that is less than an expectation value selected from a range of about 1e−2 to about 1e−9.

30. The computer program product of claim 29 wherein said expectation value is about 1e−6.

31. The computer program product of claim 21 wherein said allowed class of amino acids consists of R, K, H, D, E, S, T, and C.

32. The computer program product of claim 21 wherein said allowed class of substitution types consists of R→K, K→R, K→H, H→K, H→R, R→H, D→E, E→D, E→H, H→E, D→H, H→D, S→T, and T→S.

33. The computer program product of claim 21 wherein said threshold percentage of said set of aligned query sequences comprises about 80 percent of said set of aligned query sequences.

34. The computer program product of claim 22 wherein said three-dimensional representation of said target sequence is selected from the group consisting of a crystal structure of said target sequence, a structure of said target sequence derived by nuclear magnetic resonance, and a homology model of said target sequence.

35. The computer program product of claim 21 wherein said polar atom is selected from the group consisting of OD1, OD2, OE1, OE2, NZ, NE, NH1, NH2, ND1, NE2, SG, OH, OG, OG1, ND2, and NE2.

36. The computer program product of claim 22 wherein said threshold distance is selected from the range of about 2.0 Å to about 7.0 Å.

37. The computer program product of claim 36 wherein said threshold distance is selected from the range of about 3.0 Å to about 6.0 Å.

38. The computer program product of claim 22, the computer program mechanism causing the processor to execute the additional steps of:
adjusting one or more of said predetermined criterion, said threshold percentage, and said threshold distance; and
repeating said choosing step, said identifying step, said selecting step and said obtaining step.

39. The computer program product of claim 22, the computer program mechanism causing the processor to execute the additional steps of:
adjusting said threshold distance; and
repeating said obtaining step.

40. The computer program product of claim 22, the computer program mechanism causing the processor to execute the additional steps of:
setting said predetermined criterion, said threshold percentage, and said threshold distance;
executing said choosing step, said identifying step, said selecting step, and said obtaining step;
adjusting one or more of said predetermined criterion, said threshold percentage, and said threshold distance; and
repeating said choosing step, said identifying step, said selecting step, and said obtaining step.

41. A computer readable memory used to direct a client/server system to function in a specified manner, comprising:
an alignment module for aligning a plurality of query sequences; said alignment module including executable instructions stored in said computer readable memory, said executable instructions including:
instructions for reading a plurality of query sequences from an amino acid sequence database;
instructions for aligning said plurality of query sequences to a target sequence in order to form a set of aligned query sequences; and
instructions for choosing a subset of aligned query sequences from said set of aligned query sequences, each aligned query sequence in said subset of aligned query sequences satisfying a predetermined criterion; and
a control module for using said aligned set of query sequences to select a plurality of candidate active site positions in said target sequence, said control module including executable instructions stored in said computer readable memory, said executable instructions including:
instructions for identifying substitution types for each residue position i in said target sequence, each substitution type characterized by a difference between (i) a residue identity at a residue position i in an aligned query sequence in said set of aligned query sequences and (ii) said residue identity at said residue position i in said target sequence; and
instructions for selecting said plurality of candidate active site positions based on a sequence-based criterion that each candidate active site position in said plurality of candidate active site positions is a residue position i in said target sequence in which (i) a residue identity at said residue position i in said target sequence is in an allowed class of amino acids, (ii) each substitution type identified for said residue position i in said target sequence is in an allowed class of substitution types, and (iii) a threshold percentage of said set of aligned query sequences includes a residue that is aligned with said residue position i in said target sequence.

42. The computer readable memory of claim 41, the memory further comprising a candidate set selection module for obtaining a set of candidate active site positions from said plurality of candidate active site positions, said candidate set selection module including executable instructions stored in said computer readable memory, said executable instructions including:
instructions for selecting a set of candidate active site positions from said plurality of candidate active site positions, each candidate active site position in said set having a property that, when a first residue corresponding to a candidate active site position in said set is mapped to a three-dimensional representation of said target sequence that corresponds to said position, a polar or charged atom of a side-chain of said first residue is within a threshold distance of at least one other polar or charged atom belonging to a side-chain of a second residue, wherein said second residue corresponds to a second candidate active site position in said set that has been mapped to said three-dimensional representation.

43. The computer readable memory of claim 41 wherein, for each query sequence in said plurality of query sequences, said instructions for aligning further comprise:
instructions for positioning said query sequence and said target sequence relative to each other;
instructions for scoring a sequence similarity between said amino acid sequence and said target sequence after said positioning step; and
instructions for repeating said instructions for positioning and said instructions for scoring until said sequence similarity between said query sequence and said target sequence has been maximized or said repeating step has been executed a predetermined number of times.

44. The computer readable memory of claim 43 wherein said instructions for positioning include instructions for introducing a “phase shift” into said query sequence or said target sequence.

45. The computer readable memory of claim 43 wherein said instructions for positioning include instructions for introducing a “gap” into said query sequence or said target sequence.

46. The computer readable memory of claim 45 wherein said instructions for positioning include instructions for extending said “gap” in said query sequence or said target sequence.

47. The computer readable memory of claim 41 wherein said instructions for aligning include instructions for performing a global comparison method or a local comparison method.

48. The computer readable memory of claim 41 wherein said instructions for aligning comprise instructions for pairwise alignment of each query sequence in said plurality of query sequences to said target sequence using BLAST, PSI-BLAST, WU-BLAST-2, MEGABLAST, or FASTA; wherein an alignment scoring table used for scoring said sequence similarity between each query sequence in said plurality of query sequences to said target sequence is selected from the group consisting of Blosum62, a Dayhoff table, PAM250, a WAC matrix, a Risler matrix, and PAM120.

49. The computer readable memory of claim 48 wherein said predetermined criterion is an overall sequence similarity with said target sequence that is less than an expectation value selected from a range of about 1e−2 to about 1e−9.

50. The computer readable memory of claim 49 wherein said expectation value is about 1e−6.

51. The computer readable memory of claim 41 wherein said allowed class of amino acids consists of R, K, H, D, E, S, T, and C.

52. The computer readable memory of claim 41 wherein said allowed class of substitution types consists of R→K, K→R, K→H, H→K, H→R, R→H, D→E, E→D, E→H, H→E, D→H, H→D, S→T, and T→S.

53. The computer readable memory of claim 41 wherein said threshold percentage of said set of aligned query sequences comprises about 80 percent of said set of aligned query sequences.

54. The computer readable memory of claim 42 wherein said three-dimensional representation of said target sequence is selected from the group consisting of a crystal structure of said target sequence, a structure of said target sequence derived by nuclear magnetic resonance, and a homology model of said target sequence.

55. The computer readable memory of claim 47 wherein said polar atom is selected from the group consisting of OD1, OD2, OE1, OE2, NZ, NE, NH1, NH2, ND1, NE2, SG, OH, OG, OG1, ND2, and NE2.

56. The computer readable memory of claim 42 wherein said threshold distance is selected from the range of about 2.0 Å to about 7.0 Å.

57. The computer readable memory of claim 56 wherein said threshold distance is selected from the range of about 3.0 Å to about 6.0 Å.

58. The computer readable memory of claim 42, the computer program mechanism causing the processor to execute the additional steps of:
adjusting one or more of said predetermined criterion, said threshold percentage, and said threshold distance; and
repeating said choosing step, said identifying step, said selecting step and said obtaining step.

59. The computer readable memory of claim 42, the memory further comprising an automation module including executable instructions stored in said computer readable memory, said executable instructions including:
instructions for adjusting said threshold distance; and
instructions for repeating said instructions for selecting a set of candidate active site positions.

60. The computer readable memory of claim 42, the memory further comprising an automation module including executable instructions store in said computer readable memory, said executable instructions including:
instructions for setting said predetermined criterion, said threshold percentage, and said threshold distance;
instructions for executing said instructions for reading, said instructions for aligning, said instructions for choosing, said instructions for identifying, said instructions for selecting said plurality of candidate active site positions, and said instructions for selecting a set of candidate active site positions;
instructions for adjusting one or more of said predetermined criterion, said threshold percentage, and said threshold distance; and
instructions for repeating said instructions for choosing, said instructions for identifying, said instructions for selecting said plurality of candidate active site positions, and said instructions for selecting a set of candidate active site positions.
